(19) United States
(12) Patent Application Publication
(10) Pub. No.: US 2018/0319865 A1
(43) Pub. Date: Nov. 8, 2018

(54) Identification of Novel Small Molecule Beta2 Integrin Agonists

(71) Applicant: Rush University Medical Center, Chicago, IL (US)

(72) Inventors: Vineet GUPTA, Chicago, IL (US); Xiaobo Li, Chicago, IL (US)

(21) Appl. No.: 16/070,628

(22) PCT Filed: Jan. 20, 2017
(86) PCT No.: PCT/US17/14222

§ 371 Date: Jul. 17, 2018

Related U.S. Application Data
(60) Provisional application No. 62/286,190, filed on Jan. 22, 2016.

(51) Int. Cl. C07K 14/705; A61K 47/68; G01N 33/15

(52) U.S. Cl. CPC C07K 14/70553; A61K 47/6803; A61K 31/427; A61K 2300/00; G01N 33/15

(57) ABSTRACT

The application describes small molecules capable of modulating activity of beta2 family of integrins, such as integrin CD11b/CD18 (also known as Mac-1, CR3 and αMβ2). Such compounds may be used in certain embodiments for treating a disease or condition, such as inflammation, immune-related disorders, cancer, ischemia-reperfusion injury, stroke, neointimal thickening associated with vascular injury, wound-healing, organ transplantation and cardiovascular disease, among others.

### CROSS REFERENCE TO RELATED APPLICATIONS

This application is a U.S. national phase application of International Patent Application No. PCT/US2017/14222, filed on Jan. 20, 2017, which claims priority to U.S. Provisional Patent Application No. 62/286,190, filed on Jan. 22, 2016, each of which is incorporated herein in its entirety by express reference thereto.

### FEDERALLY SPONSORED RESEARCH

This invention was made with U.S. government support under grant number 1 R03 NS053659-01 awarded by the National Institutes of Health. The U.S. government has certain rights in the invention.

### TECHNICAL FIELD

This invention relates to compounds that modulate function of beta2 family of integrins.

### BACKGROUND

Integrins are non-covalently linked α/β heterodimeric receptors that mediate cell adhesion, migration and signaling. Together with their ligands, integrins play central roles in many processes including development, hemostasis, inflammation and immunity, and in pathologic conditions such as cancer invasion and cardiovascular disease. Leukocyte migration and recruitment is essential for their normal immune response to injury and infection and in various inflammatory and autoimmune disorders [1]. For example, in response to injury or infection leukocytes are recruited into the tissues where they participate in immune clearance [2].

The β2 integrins, a sub-family of α/β heterodimeric integrin receptors that have a common β-subunit (β2, CD18) but distinct α-subunits (CD11a, CD11b, CD11c and CD11d [3]), are leukocyte specific receptors [4]. β2 integrins, including highly expressed integrin CD11b/CD18 (also known as Mac-1, CR3 and αMβ2), modulate leukocyte functions, including cell adhesion, migration, recruitment and activation [2]. CD11b/CD18 recognizes the complement fragment iC3b, Fibrinogen, and ICAM-1 as ligands, among various others. CD11b/CD18 has been implicated in many inflammatory and autoimmune diseases, such as ischemia-reperfusion injury (including acute renal failure and atherosclerosis), tissue damage, stroke, neointimal thickening in response to vascular injury and the resolution of inflammatory processes [5-9]. Leukocytic β2 integrins also modulate tumor infiltration. For example, tumors also secrete inflammatory cytokines to recruit CD11b-expressing myeloid cells to facilitate neovascularization [10]. During cancer treatments, irradiated tumors recruit large numbers of specific leukocytes, bone marrow-derived CD11b-expressing myeloid cells expressing matrix metalloproteinase-9 (MMP-9), that restore tumor vasculature and allow tumor re-growth and recurrence [11]. Recent studies have shown that treatment with CD11b antagonists (anti-CD11b antibody) reduces CD11b-expressing myeloid cell infiltration and an enhancement of tumor response to radiation in mice [11]. Additionally, inflammatory leukocytes potentiate anti-GBM nephritis. Experimental anti-GBM nephritis in mice is a model of rapidly progressive glomerulonephritis, is characterized by proteinuria, leukocyte infiltration and glomerular crescent formation [12, 13]. Leukocytes play a critical role in the pathogenesis of anti-GBM nephritis, and their number correlates with the percentage of crescentic glomeruli. CD11b^{-/-} animals show no proteinuria and strong protection of renal function [14], suggesting that agents targeting this integrin have a potential to treat this disease.

In addition to increasing cell adhesion and modulating migration, the beta2 integrins, including CD11b/CD18, mediate a number of intracellular signaling events, including production of reactive oxygen species and modulation of a number of pro- and anti-inflammatory genes in inflammatory cells [15-20]. Integrin activation and ligand binding leads to its clustering on the cell surface and initiates outside in signaling, including the activation of PI3-K/Akt and MAPK/ERK1/2 pathways [16, 21], thereby mimicking the anchorage-dependent pro-survival signals in most cells. Ligation and clustering of integrins also synergistically potentiates intracellular signaling by other receptors (such as, Toll-like receptors (TLRs) and cytokine receptors interleukin-1 receptor (IL-1R) and TNFR) and both induce transcription factor (such as, NF-κB) dependent expression of pro-inflammatory cytokines (e.g.; IL1β, IL6, TNFα) as well as release of other factors (e.g.; Tissue Factor).

Thus, there is a considerable potential for agents that modulate the function of CD11b/CD18 and other beta2 integrin as therapeutic agents for the treatment of various diseases and conditions, including inflammatory conditions. Indeed, blocking beta2 integrins, including CD11b/CD18, and their ligands with antibodies and ligand mimics (anti-adhesion therapy) [22-24] and genetic ablation of CD11a, CD11b, CD11c or CD18 decreases the severity of inflammatory response in vivo in many experimental models [25-27]. However, such blocking agents have had little success in treating inflammatory/autoimmune diseases in humans [26, 28], perhaps because complete blockage of integrins with antibodies is difficult due to availability of a large mobilizable intracellular pool of such integrins (for example, CD11b/CD18) [29, 30] or because suppressing leukocyte recruitment with blocking agents requires occupancy of >90% of active integrin receptors [31]. Anti-integrin β2 antibodies have also shown unexpected side effects [32].

The above suggests that what is needed are small molecules that selectively regulate the ligand binding and function of beta2 integrins, including integrins CD11a/CD18, CD11b/CD18 and integrin CD11c/CD18. Additionally, agents that do not compete with ligand binding (by targeting allosteric regulatory sites, such as the hydrophobic site-for-isoleucine (SILEN) pocket in CD11b/CD18) are especially desired. Moreover, compounds and methods to enhance or promote integrin-mediated cell-adhesion are highly desired. Furthermore, compounds that regulate cellular functions (such as cell activation and signaling) of inflammatory cells are highly desirable. Integrin activation has been proposed as an alternative to blockade (anti-adhesion) for modulating cell function and treating a number of diseases, including inflammatory diseases [33, 34]. It is based on the initial finding by Harlan and co-workers over 15 years ago that freezing of integrin α4β1 in high avidity state using an activating antibody increases cell adhesion and decreases eosinophil migration [35]. Recent research with knock-in animals that express activating mutants of integrins αLβ2 [36, 37] and α4β7 [38] provides in vivo support for this hypothesis.

However, compounds that enhance integrin activity are highly desired but no integrin-specific compounds and methods have been previously described. Additionally, whether transient activation of a fraction of native receptors in vivo, as is expected from small molecule treatment, will have biological effect also remains an open question. Moreover, an important requirement of useful compounds and compositions that regulate beta2 integrins, including CD11b/CD18, is that they not negatively affect the cell, tissue and animal viability. Some have suggested that integrin agonists might induce killing of target cells (Yang et al., J Biol Chem 281, 37904 (2006)), which is not desirable.

### SUMMARY

The invention describes novel compounds, compositions and methods that are useful in targeting beta2 integrins, including CD11b/CD18. The invention also describes compositions and methods that are useful in detecting, diagnosing or treating various mammalian diseases and conditions, including, but not limited to, inflammatory diseases and conditions, autoimmune diseases and conditions and transplantation. The invention describes compositions and methods that are useful in improving the health of a patient.

The compounds and methods described in this invention are useful in activating beta2 integrins (such as CD11b/CD18), thereby modulating biological function of cells that express this protein. Such cells include leukocytes. Such biological functions include signaling pathways and gene expression. The compounds and methods described in this invention can also be used, via activation of beta2 integrin, to regulate levels of secreted factors in vitro and in vivo. In vitro or in vivo modulation of the levels of such soluble factors, which include factors such as cytokines, chemokines, microparticles, small molecules and other proteins and peptides, is also useful in treating a number of diseases and conditions in patients, including inflammatory and auto-immune disease and conditions.

This invention also describes our novel strategy, as an alternative to the anti-adhesion strategy that is currently practiced in literature, for regulating the biological function of integrins and integrin-expressing cells. Our strategy involves integrin activation, rather than its blockade, as a way to modulate the function or activity of beta2 integrins, including CD11b/CD18, and the function or activity of cells that express beta2 integrins, such as of leukocytes. The compounds of this invention are easily delivered in vitro, ex vivo and in vivo and can be readily derivatized or optimized for use in patients. Additionally, this invention shows that transient activation of a fraction of native receptors in vitro and in vivo (via administration of the compounds of this invention) affects the function of biological cells. Integrin activation is a novel, useful, pharmacologically targetable methodology to treat, without limitation, a variety of inflammatory and autoimmune diseases and conditions.

One embodiment described herein is any one of the compounds listed in Table 1 or Table 2 or a derivative, salt, or ester thereof that augment beta2 integrin activity. In one aspect described herein, the compound is listed in Table 2. In another aspect described herein, the beta2 integrin is CD11b/CD18. In another aspect described herein, the beta2 integrin is CD11b^{E320A}/CD18. In another aspect described herein, the beta2 integrin is CD11c/CD18. In another aspect described herein, the compound binds to an αA-domain of a beta2 integrin. In another aspect described herein, the compound binds to an αA-domain of a CD11b integrin. In another aspect described herein, the compound binds to an αA-domain of a CD11c integrin.

Another embodiment described herein is a pharmaceutical composition comprising one or more of the compounds of Table 1 or Table 2 and one or more pharmaceutically acceptable excipients.

Another embodiment described herein is a genus of one or more of the compounds listed in Table 2, wherein the genus is identified by quantitative structure-activity relationship experiments and biological methods.

Another embodiment described herein is a species identified by quantitative structure-activity relationship experiments and biological assays.

Another embodiment described herein is a pharmaceutical composition comprising the species of claim described herein and one or more pharmaceutically acceptable excipients.

Another embodiment described herein is a method for identifying agonist compounds of beta2 integrin, the method comprising: (a) contacting cells with a compound on a substrate treated with fibrinogen; (b) physically repositioning the substrate such that non-adherent cells move away from the substrate by the action of gravity; and (c) detecting adherent cells on the substrate. One aspect described herein is a method for identifying agonist compounds of beta2 integrin, further comprising the step of: (d) quantifying the adherent cells on the substrate. In another aspect described herein, the cells are K562 cells expressing integrin CD11b/CD18, CD11b^{E320A}/CD18, or CD11c/CD18. In another aspect described herein, the solution further comprises Mg²⁺, Ca²⁺, or a mixture thereof. In another aspect described herein, the method further comprises removing the solution from the substrate after physically repositioning the substrate.

In another aspect described herein, removing the solution from the substrate comprises contacting adhered cells with a fixative and washing the substrate. In another aspect described herein, the fixative comprises formaldehyde. In another aspect described herein, the method is conducted without contacting the substrate with an additional liquid to wash or rinse the substrate. In another aspect described herein, detecting adherent cells comprises measuring the viability of the adherent cells or imaging the adherent cells.

Another embodiment described herein is a beta2 integrin agonist compound identified by the methods described herein. In one aspect described herein, the compound is listed in Table 2.

Another embodiment described herein is a compound of Table 2, or a derivative thereof, useful in modulating the levels of cell-secreted factors in vitro or in vivo, wherein the secreted factor comprises inflammatory cytokines or chemokines.

Another embodiment described herein is a pharmaceutical composition comprising a compound of Table 2, or a derivative thereof, useful in treating a disease or condition in a subject.

Another embodiment described herein is a pharmaceutical composition comprising a compound of Table 2, or a derivative thereof, useful in detecting or diagnosing a disease or condition in a subject.

Another embodiment described herein is a method of improving health of a subject, comprising administering to a subject an effective amount of any compound of Table 2, or a derivative thereof.

Another embodiment described herein is a method of improving health of a subject, comprising administering to a subject a pharmaceutical composition comprising an effective amount of any compound of Table 2, or a derivative thereof.

### BRIEF DESCRIPTION OF THE DRAWINGS

FIG. 1 shows a schematic illustration of the design of a high throughput-screening assay.

### DETAILED DESCRIPTION

One aspect of the invention relates to compounds described in this invention, their derivatives, pharmaceutically acceptable salts or hydrates thereof.

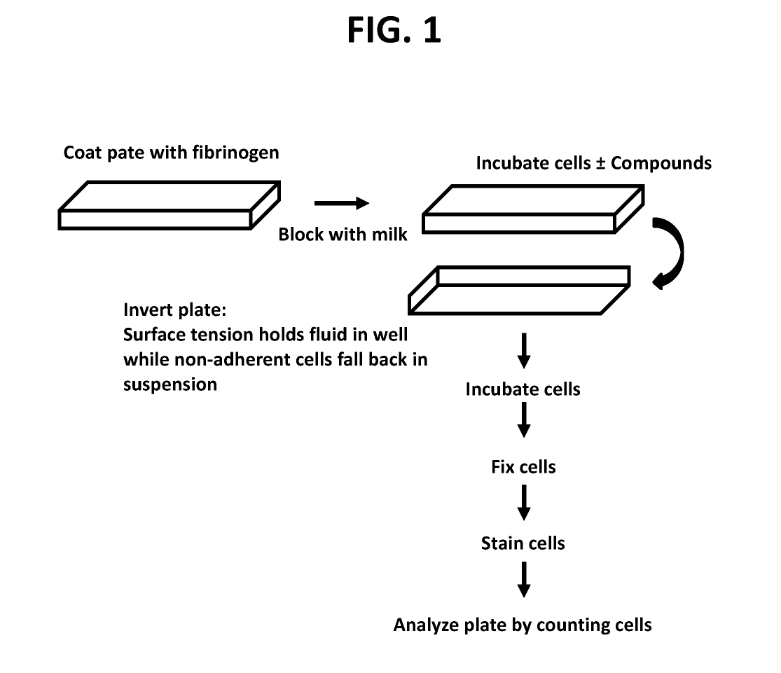

One aspect of the invention relates to the compounds listed in FIG. 1, their derivatives or pharmaceutically acceptable salts thereof.

In certain embodiments, the invention relates to a pharmaceutical composition comprising a compound of the invention or a pharmaceutically acceptable salt thereof and a pharmaceutically acceptable diluent, carrier, excipient or adjuvant.

In certain embodiments, the invention relates to a pharmaceutical composition comprising a compound of the invention or a pharmaceutically acceptable salt thereof in combination with another compound or agent to modulate or treat a condition and a pharmaceutically acceptable diluent, carrier, excipient or adjuvant.

Beta2 family integrins, such as CD11b/CD18, are known targets in a number of mammalian diseases and conditions and there is considerable potential for agents that modulate the function of these integrins as therapeutic agents for the treatment of various mammalian diseases and conditions, including inflammatory conditions. Indeed, many agents that block binding of these integrins to their ligands (antagonists) have been previously described in the literature. However, such blocking agents have had little success in treating inflammatory/autoimmune diseases in humans [26, 28], and some have shown unexpected side effects [32].

In certain aspects, the compounds of this invention, and their derivatives, are useful in promoting the function or activity of beta2 integrins, including CD11b/CD18. In certain aspects, the mechanism of action of the described compounds includes a direct binding of the compounds to the ligand binding αA- (or αI-) domain of beta2 integrins in a high-affinity conformation. In certain aspects, binding by the compounds of this invention to the αA-domain of beta2 integrins leads to a conversion of the αA-domain from an inactive to an active, ligand-competent conformation. In certain aspects, the binding by the compounds of this invention to the αA-domain of beta2 integrins leads to stabilization of the αA-domain into an active, ligand-competent conformation. In certain other aspects, the compounds of this invention bind very weakly to the αA-domain of beta2 integrins, when the αA-domain is in an inactive conformation. In certain aspects, binding by the compounds of this invention to beta2 integrins leads to a conversion of the integrin molecule from an inactive to an active, ligand-competent conformation. In certain aspects, the compounds do not compete with ligands for binding to their target beta2 integrins, bind in an allosteric pocket of the protein and allosterically regulate the protein function. Furthermore, in certain aspects, the compounds of this invention are not ligand mimics.

In one aspect of this invention, the compounds bind, with varying binding affinities, to all members of the beta2 integrin family, including CD11a/CD18, CD11b/CD18, CD11c/CD18 and CD11d/CD18. Thus, these compounds are useful in targeting all beta2 integrins and that the utility of these compounds and methods is not limited to a single type of integrin.

In certain aspects, integrin activation by the described compounds increases adhesion of integrin-expressing cells to extra-cellular matrix, ligands or other targets. In certain aspects, increased cell adhesivity reduces the lateral motility of cells (including cellular chemotaxis). In certain aspects, increased cell adhesivity reduces the transendothelial migration (TEM) of cells. In certain aspects, the compositions and methods described herein affect leukocyte recruitment. They can achieve this, for example, by increasing leukocyte slow rolling and adhesivity to the inflammed endothelium, which could be reversed with a blocking antibody. In certain aspects, the compositions and methods described herein reduce the levels of secreted factors. Such factors include, without limitation, inflammatory factors, for example TNFalpha, IL1 beta, IL-6, IFNgamma, soluble uPAR, microparticles among others. In a related aspect, the compositions and methods described herein reduce the levels of secreted factors by integrin-expressing cells. In another aspect, the compositions and methods described herein reduce the levels of secreted factors by cells that interact with beta2 integrin expressing cells. In certain aspects, the compositions and methods described herein increase the level of secreted factors. Such factors include anti-inflammatory factors, for example IL-10 among others. In certain aspects, the compositions and methods described herein modify the signaling pathways in cells. In certain aspects, the compositions and methods described herein modify intracellular signaling pathways in beta2 integrin-expressing cells (including leukocytes, among others). Such pathways include, without limitation, the NF-κB pathway, AKT pathway, MAPK pathway, Toll-like receptor signaling pathway, cytokine receptor signaling pathways, among others. In certain aspects, the compounds and methods of this invention activate beta2 integrins, which induces intracellular signaling that synergizes or opposes other signaling pathways in the cells. In certain aspects, the compositions and methods described herein modify the signaling pathways in cells that interact with the beta2-integrin expressing cells. Such cells include other leukocyte subsets, lymphocytes, endothelial cells, among others. In certain other aspects, the compositions and methods described herein modify the signaling pathways in cells that interact with factors secreted by the beta2-integrin expressing cells (such as leukocytes, lymphocytes, endothelial cells, among others).

In certain embodiments, the compounds of the invention regulate the function of beta2 integrins, especially integrin CD11b/CD18.

In certain embodiments, the compounds of the invention regulate conformation of beta2 integrins, especially integrin CD11b/CD18.

In certain embodiments, the compounds of the invention regulate the organization of beta2 integrins in a cell, especially integrin CD11b/CD18. In certain such embodiments, the organization of beta2 integrins includes its dimerization or multimerization with itself or other proteins and substances.

In certain embodiments, the compounds of the invention regulate the organization of beta2 integrins on a cell membrane, especially integrin CD11b/CD18. In certain such embodiments, the organization of beta2 integrins includes its dimerization or multimerization with itself or other proteins and substances.

In certain embodiments, the compounds of the invention increase the binding of beta2 integrins, especially integrin CD11b/CD18, to its ligands.

In certain embodiments, the compounds of the invention increase the binding of beta2 integrins, especially integrin CD11b/CD18, to its ligands, wherein the ligand is independently selected from ICAM-1, ICAM-2, ICAM-3, iC3b, fibrinogen, Factor X, fibrin, uPAR and GP Ibalpha.

In certain embodiments, the compounds of the invention increase the binding of beta2 integrins, especially integrin CD11b/CD18, to its ligands, wherein binding of the compound with the protein modulates at least one function normally associated with binding of a natural ligand of the protein. In certain embodiments, the function is independently selected from the group consisting of rolling of leukocytes with vascular endothelium, binding of leukocytes with vascular endothelium, crawling of leukocytes with vascular endothelium, translocation of leukocytes through vascular endothelium, infiltration of leukocytes into intimal tissue, release of one or more soluble factors from leukocytes, release of a chemotactic factor from leukocytes, release of a growth factor from leukocytes, leukocyte-binding-associated release of a chemotactic factor from a tissue, leukocyte-binding-associated release of a growth factor from a tissue, leukocyte-binding-associated release of one or more soluble factors from a tissue, change in the level of one or more soluble factors in circulation and change in the level of one or more insoluble factors.

In certain embodiments, the compounds of the invention modulate function of cells in vitro or in vivo. In certain such embodiments, the function is independently selected from the group consisting of rolling of leukocytes with vascular endothelium, binding of leukocytes with vascular endothelium, crawling of leukocytes with vascular endothelium, translocation of leukocytes through vascular endothelium and infiltration of leukocytes into intimal tissue. In certain other embodiments, the function is independently selected from the group consisting of release of one or more secreted factors from leukocytes, release of a chemotactic factor from leukocytes, release of a growth factor from leukocytes, leukocyte-binding-associated release of a chemotactic factor from a tissue, leukocyte-binding-associated release of a growth factor from a tissue, leukocyte-binding-associated release of one or more soluble factors from a tissue, change in the level of one or more soluble factors in circulation and change in the level of one or more insoluble factors. In certain such embodiments, the secreted factors include cytokines. In certain such embodiments, the cytokines include pro-inflammatory cytokines. In certain other embodiments, the cytokines include anti-inflammatory cytokines. In certain other embodiments, the cytokines include, but not limited to, IL-1 beta, IL-6 and IL-10. In certain other embodiments, the soluble factors include, but not limited to, TNFalpha and interferon gamma.

In certain embodiments, the compounds of the invention modulate biological function in vitro or in vivo. In certain such embodiments, the biological function is independently selected from the group consisting of gene expression, epigenetic profile, protein expression, protein levels, protein modifications, post-translational modifications and signaling. In certain such embodiments, the compounds of the invention modulate biological function in leukocytes. In certain other embodiments, the compounds of the invention modulate biological function in other cells. In certain other embodiments, the compounds of the invention modulate biological function in tissues.

In certain embodiments, the invention relates to methods for the regulation of the function of beta2 integrins, especially integrin CD11b/CD18, comprising administering a compound of the invention.

In certain embodiments, the invention relates to methods for the regulation of the conformation of beta2 integrins, especially integrin CD11b/CD18, comprising administering a compound of the invention.

In certain embodiments, the invention relates to methods for the regulation of the organization of beta2 integrins in a cell, especially integrin CD11b/CD18, comprising administering a compound of the invention. In certain such embodiments, the organization of beta2 integrins includes is dimerization or multimerization with itself or other proteins and substances.

In certain embodiments, the invention relates to methods for the regulation of the organization of beta2 integrins on a cell membrane, especially integrin CD11b/CD18, comprising administering a compound of the invention. In certain such embodiments, the organization of beta2 integrins includes is dimerization or multimerization with itself or other proteins and substances.

In certain embodiments, the invention relates to methods for increasing the binding of beta2 integrins, especially integrin CD11b/CD18, to its ligands comprising administering a compound of the invention.

In certain embodiments, the invention relates to methods for increasing the binding of beta2 integrins, especially integrin CD11b/CD18, to its ligands comprising administering a compound of the invention, wherein the ligand is independently selected from ICAM-1, ICAM-2, ICAM-3, iC3b, fibrinogen, Factor X, fibrin, uPAR and GP Ibalpha.

In certain embodiments, the invention relates to methods for increasing the binding of beta2 integrins, especially integrin CD11b/CD18, to its ligands comprising administering a compound of the invention, wherein binding of the compound with the protein modulates at least one function normally associated with binding of a natural ligand of the protein. In certain embodiments, the function is independently selected from the group consisting of rolling of leukocytes with vascular endothelium, binding of leukocytes with vascular endothelium, crawling of leukocytes with vascular endothelium, translocation of leukocytes through vascular endothelium, infiltration of leukocytes into intimal tissue, release of one or more soluble factors from leukocytes, release of a chemotactic factor from leukocytes, release of a growth factor from leukocytes, leukocyte-binding-associated release of a chemotactic factor from a tissue, leukocyte-binding-associated release of a growth factor from a tissue, leukocyte-binding-associated release of one or more soluble factors from a tissue, change in the level of one or more soluble factors in circulation and change in the level of one or more insoluble factors.

In certain embodiments, the invention relates to methods for modulating function of cells in vitro or in vivo comprising of administering a compound of the invention. In certain such embodiments, the function is independently selected from the group consisting of rolling of leukocytes with vascular endothelium, binding of leukocytes with vascular endothelium, crawling of leukocytes with vascular endothelium, translocation of leukocytes through vascular endothelium and infiltration of leukocytes into intimal tissue. In certain other embodiments, the function is independently selected from the group consisting of release of one or more soluble factors from leukocytes, release of a chemotactic factor from leukocytes, release of a growth factor from leukocytes, leukocyte-binding-associated release of a chemotactic factor from a tissue, leukocyte-binding-associated release of a growth factor from a tissue, leukocyte-binding-associated release of one or more secreted factors from a tissue, change in the level of one or more soluble factors in circulation and change in the level of one or more insoluble factors. In certain such embodiments, the soluble factors include cytokines. In certain such embodiments, the cytokines include pro-inflammatory cytokines. In certain other embodiments, the cytokines include anti-inflammatory cytokines. In certain other embodiments, the cytokines include, but not limited to, IL-1beta, IL-6 and IL-10. In certain other embodiments, the soluble factors include, but not limited to, TNFalpha and interferon gamma.

In certain embodiments, the invention relates to methods for modulating biological function in vitro or in vivo comprising of administering a compound of the invention. In certain such embodiments, the biological function is independently selected from the group consisting of gene expression, epigenetic profile, protein expression, protein levels, protein modifications, post-translational modifications and signaling. In certain such embodiments, the compounds of the invention modulate biological function in leukocytes. In certain other embodiments, the methods of the invention modulate biological function in other cells. In certain other embodiments, the methods of the invention modulate biological function in tissues.

In certain aspects, the invention includes compositions and methods to optimize the in vivo half-life of the compounds of this invention, or their derivatives.

In certain embodiments, the invention relates to a composition for use on an article, such as a catheter or a stent, comprising administering an effective amount of any compound of the invention, or a derivative thereof, to the article. In certain embodiments, the invention relates to a composition for use on an article for a patient, comprising administering an effective amount of any compound of the invention, or a derivative thereof, to the article.

In certain embodiments, the invention relates to a drug-eluting stent media; wherein said drug-eluting stent media comprises a pharmaceutical composition; wherein said pharmaceutical composition comprises a compound of the invention or a pharmaceutically acceptable salt thereof and optionally a pharmaceutically acceptable diluent, carrier, excipient or adjuvant.

In certain embodiments, the invention relates to a composition comprising a drug-eluting stent media; wherein said drug-eluting stent media comprises a pharmaceutical composition; wherein said pharmaceutical composition comprises a compound of the invention or a pharmaceutically acceptable salt thereof and optionally a pharmaceutically acceptable diluent, carrier, excipient or adjuvant.

In certain embodiments, the invention relates to a method of improving health of a patient, comprising administering an article comprising an effective amount of any compound of the invention, or a derivative thereof, to the patient.

In certain embodiments, the invention relates to a compound or method of improving health of a patient, comprising administering an article comprising an effective amount of any compound of the invention, or a derivative thereof, to a cell, tissue or organ of the patient, wherein the administration is performed in vivo or ex vivo.

In certain embodiments, the compounds and methods of this invention have no systemic vascular toxicity and do not induce injury or apoptosis in vascular cells.

In certain embodiments, the invention relates to a pharmaceutical composition useful in treating a disease or condition associated with the activity of beta2 integrins. In certain embodiments, such a disease or condition is selected from inflammation (including, but not limited to, acute and chronic inflammation), inflammatory skin diseases, immune-related disorders, autoimmune diseases, burn, immune deficiency, acquired immune deficiency syndrome (AIDS), myeloperoxidase deficiency, Wiskott-Aldrich syndrome, chronic kidney disease, chronic granulomatous disease, hyper-IgM syndromes, leukocyte adhesion deficiency, iron deficiency, Chediak-Higashi syndrome, severe combined immunodeficiency, diabetes, obesity, hypertension, HIV, wound-healing, remodeling, scarring, fibrosis, stem cell therapies, cachexia, encephalomyelitis, multiple sclerosis, psoriasis, lupus, rheumatoid arthritis, immune-related disorders, radiation injury, transplantation, cell transplantation, cell transfusion, organ transplantation, organ preservation, cell preservation, asthma, irritable bowel disease, irritable bowel syndrome, ulcerative colitis, colitis, bowel disease, cancer, leukemia, ischemia-reperfusion injury, stroke, neointimal thickening associated with vascular injury, bullous pemphigoid, neonatal obstructive nephropathy, familial hypercholesterolemia, atherosclerosis, dyslipidemia, aortic aneurisms, arteritis, vascular occlusion, including cerebral artery occlusion, complications of coronary by-pass surgery, myocarditis, including chronic autoimmune myocarditis and viral myocarditis, heart failure, including chronic heart failure (CHF), cachexia of heart failure, myocardial infarction, stenosis, restenosis after heart surgery, silent myocardial ischemia, post-implantation complications of left ventricular assist devices, thrombophlebitis, vasculitis, including Kawasaki's vasculitis, giant cell arteritis, Wegener's granulomatosis, traumatic head injury, post-ischemic-reperfusion injury, post-ischemic cerebral inflammation, ischemia-reperfusion injury following myocardial infarction and cardiovascular disease, and wherein the pharmaceutical composition comprises a pharmaceutically acceptable carrier, diluent or excipient and a compound of the invention. In certain such embodiments, the invention relates to a pharmaceutical composition useful in treating a disease or condition associated with the activity of beta2 integrins such as inflammatory kidney disease, a condition that affects millions of people in the world and leads to renal failure, and restenosis, a common problem in people who have undergone angioplasty, one of the most common procedures in interventional cardiology. In certain such embodiments, the beta2 integrin is CD11b/CD18.

In one aspect, the compounds and methods of this invention are useful in treating cancer or reducing tumors in patients. In one related aspect, the compounds and methods of this invention modulate tumor infiltration of leukocytes. For example, tumors also secrete inflammatory cytokines to recruit cells expressing beta2 integrins, such as CD11b/CD18, to facilitate neovascularization [10]. During cancer treatments, including via chemotherapy and irradiation, tumors recruit large numbers of specific leukocytes or bone marrow-derived cells that restore tumor vasculature and allow tumor re-growth and recurrence [11]. In one aspect, the compounds and methods of this invention are useful in reducing activity, such as infiltration, of such cells. In another aspect, the compounds and methods of this invention are useful in enhancing the response of other cancer treatments, such as chemotherapy, antibody therapy and irradiation [11].

In certain embodiments, the invention relates to methods for treating a disease or condition selected from inflammation (including, but not limited to, acute and chronic inflammation), inflammatory skin diseases, immune-related disorders, autoimmune diseases, burn, immune deficiency, acquired immune deficiency syndrome (AIDS), myeloperoxidase deficiency, Wiskott-Aldrich syndrome, chronic kidney disease, chronic granulomatous disease, hyper-IgM syndromes, leukocyte adhesion deficiency, iron deficiency, Chediak-Higashi syndrome, severe combined immunodeficiency, diabetes, obesity, hypertension, HIV, wound-healing, remodeling, scarring, fibrosis, stem cell therapies, cachexia, encephalomyelitis, multiple sclerosis, psoriasis, lupus, rheumatoid arthritis, immune-related disorders, radiation injury, transplantation, cell transplantation, cell transfusion, organ transplantation, organ preservation, cell preservation, asthma, irritable bowel disease, irritable bowel syndrome, ulcerative colitis, colitis, bowel disease, cancer, leukemia, ischemia-reperfusion injury, stroke, neointimal thickening associated with vascular injury, bullous pemphigoid, neonatal obstructive nephropathy, familial hypercholesterolemia, atherosclerosis, dyslipidemia, aortic aneurisms, arteritis, vascular occlusion, including cerebral artery occlusion, complications of coronary by-pass surgery, myocarditis, including chronic autoimmune myocarditis and viral myocarditis, heart failure, including chronic heart failure (CHF), cachexia of heart failure, myocardial infarction, stenosis, restenosis after heart surgery, silent myocardial ischemia, post-implantation complications of left ventricular assist devices, thrombophlebitis, vasculitis, including Kawasaki's vasculitis, giant cell arteritis, Wegener's granulomatosis, traumatic head injury, post-ischemic-reperfusion injury, post-ischemic cerebral inflammation, ischemia-reperfusion injury following myocardial infarction and cardiovascular disease, comprising administering a compound of the invention.

Leukocyte recruitment precedes neointima formation and restenosis following percutaneous transluminal coronary angioplasty (PTCA) [5]. Denudation of the endothelial cell lining at the site of mechanical vascular injury leads to the deposition of fibrin and platelets, where selective binding between the platelet cell surface receptor GP Ibα and CD11b/CD18 expressed on the surface of leukocytes mediates the recruitment of leukocytes [40]. In certain aspects, the compositions and methods described herein decrease leukocyte recruitment upon injury, inflammation or other disease and condition in mammals. In certain aspects, the compositions and methods described herein reduce organ injury, including neointimal hyperplasia upon arterial injury. In certain other aspects, the compositions and methods described herein preserved organ function upon acute organ injury, such as ischemia-reperfusion injury. For example, the compounds preserve kidney function upon acute kidney injury. In certain aspects, the compositions and methods described herein preserved kidney function upon glomerular nephritis or nephrosis. In certain aspects, the compositions and methods described herein modulate the function of inflammatory cells, such as lymphocytes and leukocytes. For example, the compositions and methods described herein induce graft tolerance in the recipient animal. Similarly, the compositions and methods described herein reduce graft-versus-host disease in the recipient animal. Thus, in certain aspects, the compositions and methods described herein improve transplantation outcomes.

In one aspect, the compounds and methods of this invention are useful in inducing graft tolerance in patients. In one related aspect, the grafts include bone marrow, bone marrow cells, stem cells, immune cells, engineered cells, organs, tissues or other cells. In another related aspect, the grafts include one or more of bone marrow, bone marrow cells, stem cells, immune cells, engineered cells, organs, tissues or other cells.

In certain embodiments, the invention relates to a method of preventing or treating beta2 integrin (such as CD11b/CD18) mediated condition or disease in a patient comprising administering to said patient a therapeutically effective amount of a substantially pure and pharmaceutically acceptable compound of the invention.

In certain embodiments, the invention relates to a method of preventing or treating beta2 integrin (such as CD11b/CD18) expressing cell mediated condition or disease in a patient comprising administering to said patient a therapeutically effective amount of a substantially pure and pharmaceutically acceptable compound of the invention.

In certain embodiments, the invention relates to a method of detecting or diagnosing a beta2 integrin (such as CD11b/CD18) mediated condition or disease in a patient comprising administering to said patient a therapeutically effective amount of a substantially pure and pharmaceutically acceptable compound of the invention.

In certain embodiments, the invention relates to a method of detecting or diagnosing a beta2 integrin (such as CD11b/CD18) expressing cell mediated condition or disease in a patient comprising administering to said patient a therapeutically effective amount of a substantially pure and pharmaceutically acceptable compound of the invention.

In certain embodiments, the invention relates to the use of one or more compounds of the invention in an assay for the identification of modulators of beta2 integrins, especially CD11b/CD18.

In certain embodiments, the invention relates to the use of the described compounds in identification of sites and domains in beta2 integrins, especially integrin CD11b/CD18, CD11c/CD18, CD11d/CD18 and CD11a/CD18, that modulate activity of the said integrin. In certain other embodiments, the invention relates to the use of the described compounds in identification of related compounds that show selective binding for one or more of the beta2 integrins over other integrins.

In certain embodiments, the invention relates to the use of the described compounds in determining exact three-dimensional structure of the binding pocket in the target proteins, which can be used to derive more selective and/or potent binders. For example, a complex of CD11b/CD18 with a compound can be prepared and analyzed, e.g., by x-ray crystallography, nuclear magnetic resonance, or other suitable means, to identify the binding site of CD11b/CD18 that interacts with the compound.

In certain embodiments, computer-based modeling algorithms can be used to analyze the structures and conformations of compounds that bind beta2 integrins, especially CD11b/CD18, to identify structural features that contribute to successful binding. In certain embodiments, such information is analyzed in conjunction with information about the structure or conformation of CD11b/CD18 or a binding pocket thereof, such as structural information obtained by analysis of CD11b/CD18 using analytical techniques such as x-ray crystallography or nuclear magnetic resonance, to analyze interactions between binding compounds and the binding pocket they interact with. Such analysis can be used to predict the portion of, for example, CD11b/CD18 that interacts with the compound, to select compounds that possess structural features correlated with desired binding activity from a library of test compounds, or to design structures that are expected to exhibit binding with, for example, CD11b/CD18 for testing in vivo or in vitro using assays as described herein.

In certain embodiments, computer-based modeling algorithms can be used to identify novel compounds that bind beta2 integrins, especially CD11b/CD18, using structural features of the compounds of this invention. In certain embodiments, the methods used include scaffold hopping. In certain embodiments, the methods used include atom replacement, residue replacement and/or molecule replacement. In certain embodiments, such information is analyzed in conjunction with information about the structure or conformation of CD11b/CD18 or a binding pocket thereof, such as structural information obtained by analysis of CD11b/CD18 using analytical techniques such as x-ray crystallography or nuclear magnetic resonance, to analyze interactions between binding compounds and the binding pocket they interact with. Such analysis can be used to predict the portion of CD11b/CD18 that interacts with the compound, to select compounds that possess structural features correlated with desired binding activity from a library of test compounds, or to design structures that are expected to exhibit binding with CD11b/CD18 for testing in vivo or in vitro using assays as described herein.

In certain embodiments, the compounds of the invention occupy a binding pocket in αA-domain. In certain such embodiments, the αA-domain is from CD11b/CD18. In certain other embodiments, the αA-domain is from CD11aCD18. In yet other embodiments, the αA-domain is from CD11c/CD18. In yet another embodiment, the αA-domain is from CD11d/CD18. In certain embodiments, the compounds of the invention occupy a binding pocket in αA-domain. In certain such embodiments, the compounds of the invention interact with polar residues of the amino acids of αA-domain. In certain other embodiments, the compounds of the invention interact with polar side-chains of the amino acids of αA-domain. In certain embodiments, the compounds of the invention interact with residue lysine 166 of αA-domain. In certain such embodiments, the polar end of the compounds of the invention interacts with residue lysine 166 of αA-domain. In certain embodiments, the compounds of the invention interact with residue lysine 168 of αA-domain. In certain such embodiments, the polar end of the compounds of the invention interacts with residue lysine 168 of αA-domain. In certain embodiments, the non-polar end of the compounds of this invention occupies a hydrophobic pocket in the binding site in αA-domain. In certain embodiments, the polar end of the compounds of this invention occupies a pocket in the binding site in αA-domain, such that the polar end is more exposed to the solvent.

In certain embodiments, compounds of the invention are useful in enhancing the function of beta2 integrins, especially integrin CD11b/CD18. In certain embodiments, compounds of the invention are useful in promoting activation of beta2 integrins, especially CD11b/CD18, by binding to the αA-domain of the protein. In certain embodiments, compounds of the invention are useful in modulating the function of beta2 integrin expressing cells, especially leukocytes. In certain embodiments, compounds of the invention are useful in treating a disease or condition, wherein the composition modulates the function of beta2 integrins, especially CD11b/CD18. In certain embodiments, compounds of the invention are useful in detecting or diagnosing a disease or condition, wherein the compound modulates the function of beta2 integrins, especially CD11b/CD18. In certain embodiments, compounds of the invention are useful on an article for a patient, comprising administering an effective amount of any compound of this invention, or a derivative thereof, to the article.

In certain embodiments, the invention relates to methods for modulating an immune response in a patient, comprising administering to the patient, in vivo or ex vivo, an effective amount of a compound of this invention, or a derivative thereof. In certain embodiments, the invention relates to methods for improving health of a patient, comprising administering to the patient, in vivo or ex vivo, an effective amount of a compound of this invention, or a derivative thereof. In certain embodiments, the invention relates to methods for modulating function of beta2 integrins, especially CD11b/CD18, comprising administering to the integrin expressing cell an effective amount of any compound of the invention, or a derivative thereof. In certain embodiments, the invention relates to methods for modulating function or activity of beta2 expressing cells, especially leukocytes, in vitro or in vivo, comprising administering to the integrin expressing cell an effective amount of any compound of the invention, or a derivative thereof. In certain embodiments, the invention relates to methods for modulating levels of secreted factors, in vitro or in vivo, comprising administering an effective amount of any compound of the invention, or a derivative thereof. In certain embodiments, the invention relates to methods for modulating organ function in a patient, comprising administering an effective amount of any compound of the invention, or a derivative thereof, in vivo or ex vivo. In certain embodiments, the invention relates to methods for improving health of a patient, comprising administering an article comprising an effective amount of any compound of the invention, or a derivative thereof. In certain embodiments, the invention relates to methods for detecting or diagnosing a disease or condition of a patient, comprising administering to the patient an effective amount of any compound of the invention, or a derivative thereof.

In certain embodiments, the invention relates to methods for the modulation of integrin beta2, especially CD11b/CD18, comprising administering a compound of the invention. In certain such embodiments, the compound of the invention is a compound listed in Table 1 or in Table 2. In certain preferred embodiments, the invention relates to a method for modulating integrin CD11b/CD18 comprising administering a compound selected from compounds listed in Table 1 or in Table 2.

In certain embodiments, the invention relates to methods for agonizing beta2 integrins, especially integrin CD11b/CD18, comprising administering a compound of the invention. In certain such embodiments, the compound of the invention is a compound listed in Table 1. In certain such embodiments, the compound of the invention is selected from the compounds listed in Table 2.

Certain compounds of the present invention may exist in particular geometric or stereoisomeric forms. The present invention contemplates all such compounds, including cis- and trans-isomers, R- and S-enantiomers, diastereomers, (d)-isomers, (l)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts may be formed with an appropriate optically active acid or base, followed by resolution of the diastereomers thus formed by fractional crystallization or chromatographic means well known in the art, and subsequent recovery of the pure enantiomers.

The present invention includes radiolabeled forms of compounds of the invention, for example, compounds of the invention labeled by incorporation within the structure ³H or ¹⁴C or a radioactive halogen such as ¹²⁵I.

The present invention also includes labeled forms of compounds of the invention, for example, compounds of the invention labeled by linking the compound structure with biotin, with the help of a linker.

In certain embodiments, compounds of the invention, or derivatives thereof, can be used to detect or diagnose a condition or disease in a patient. In certain such embodiments, the compound of the invention is a compound of any one of the compounds listed in Table 1. In certain such embodiments, the compound is selected from the compounds listed in Table 2.

In certain embodiments, compounds and compositions of the invention, or derivatives thereof, can be used in detecting or diagnosing an inflammatory disease or condition or an autoimmune disease or condition, comprising administering a compound of the invention or a derivative thereof, where the compound binds beta2 integrins, especially CD11b/CD18. In certain such embodiments, compounds and compositions of the invention, or derivatives thereof, preferably bind to active form of beta2 integrins, especially CD11b/CD18.

In certain embodiments, the invention relates to methods for detecting or diagnosing a condition or disease in a patient comprising of administering a compound of the invention, or a derivative thereof, to the patient. In certain embodiments, the invention relates to methods for the identification of compounds or agents that modulate beta2 integrins, especially integrin CD11b/CD18. In certain embodiments, the invention relates to methods for the identification of biological function of compounds or agents that modulate integrin CD11b/CD18. In certain embodiments, the method includes a cell-adhesion-based high-throughput screening assay. In certain embodiments, the methods include in vitro and in vivo assays as described herein. While such methods and assays are demonstrated herein for identifying agonists of integrin CD11b/CD18, such methods and assays can be employed to identify compounds that inhibit or enhance cell adhesion mediated by other mechanisms as well, as will be recognized by those of skill in the art.

The term “a cell” as used herein includes a plurality of cells. Administering a compound to a cell includes in vivo, ex vivo, and in vitro administration.

To “enhance” or “promote” a function or activity, such as binding of integrin to its ligand or adhesion of cell to matrix or cell proliferation, is to increase the function or activity when compared to otherwise same conditions except for a condition or parameter of interest, or alternatively, as compared to another condition(s).

The term “modulate” as used herein includes the inhibition or suppression of a function or activity (such as cell proliferation) as well as the enhancement of a function or activity.

The term “agonist” is art-recognized. In certain embodiments, the term includes compounds and compositions that enhance or promote a function or activity (such as integrin binding to its ligand or conversion of integrin from inactive state to active state or phosphorylation of an intracellular protein).

The term “secreted factor” is art-recognized. In certain embodiments, the term includes proteins, peptides, small molecules, ions, lipids and microparticles that are released by a cell. In certain related embodiments, the term includes cytokines, chemokines, small molecules (such as cyclic AMP) and ions that are released by a cell.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use. Each carrier must be “acceptable” in the sense of being compatible with other ingredients of the formulation and not injurious to the patient.

Some examples of materials which can serve as pharmaceutically acceptable carriers include (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically acceptable salt” means an acid addition salt or a basic addition salt that is suitable for or compatible with the treatment of patients.

The term “pharmaceutically acceptable acid addition salt” as used herein means any non-toxic organic or inorganic salt of any base compounds represented by Formula I or II. Illustrative inorganic acids that form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Both the mono or di-acid salts can be formed, and such salts may exist in either a hydrated, solvated or substantially anhydrous form. In general, the acid addition salts of compounds of Formula I or II are more soluble in water and various hydrophilic organic solvents, and generally demonstrate higher melting points in comparison to their free base forms. The selection of the appropriate salt will be known to one skilled in the art. Other non-pharmaceutically acceptable salts, e.g. oxalates, may be used, for example, in the isolation of compounds of Formula I or II for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or II or any of their intermediates. Illustrative inorganic bases that form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the magnitude of, or alternatively delaying, pain sensations experienced by subjects in a treated population versus an untreated control population.

The term “solvate” as used herein means a compound of Table 1 or Table 2, or a pharmaceutically acceptable salt of a compound of Table 1 or Table 2, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate.”

As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The compositions containing the compounds of the invention can be prepared by known methods for the preparation of pharmaceutically acceptable compositions that can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the substances in association with one or more pharmaceutically acceptable vehicles or diluents, and contained in buffered solutions with a suitable pH and iso-osmotic with the physiological fluids.

The compounds of this invention may be used in the form of the free base, in the form of salts, solvates and as hydrates. All forms are within the scope of the invention. Acid addition salts may be formed and provide a more convenient form for use; in practice, use of the salt form inherently amounts to use of the base form. The acids which can be used to prepare the acid addition salts include preferably those which produce, when combined with the free base, pharmaceutically acceptable salts, that is, salts whose anions are non-toxic to the animal organism in pharmaceutical doses of the salts, so that the beneficial properties inherent in the free base are not vitiated by side effects ascribable to the anions. Although pharmaceutically acceptable salts of the basic compounds are preferred, all acid addition salts are useful as sources of the free base form even if the particular salt per se is desired only as an intermediate product as, for example, when the salt is formed only for the purposes of purification and identification, or when it is used as an intermediate in preparing a pharmaceutically acceptable salt by ion exchange procedures.

Pharmaceutically acceptable salts within the scope of the invention include those derived from the following acids; mineral acids such as hydrochloric acid, sulfuric acid, phosphoric acid and sulfamic acid; and organic acids such as acetic acid, citric acid, lactic acid, tartaric acid, malonic acid, methanesulfonic acid, ethanesulfonic acid, benzenesulfonic acid, p-toluenesulfonic acid, cyclohexylsulfamic acid, quinic acid, and the like.

In accordance with the methods of the invention, the described compounds, salts, or solvates thereof may be administered to a patient in a variety of forms depending on the selected route of administration, as will be understood by those skilled in the art. The compositions of the invention may be administered orally or parenterally. Parenteral administration includes intravenous, intraperitoneal, subcutaneous, intramuscular, transepithelial, nasal, intrapulmonary, intrathecal, rectal and topical modes of administration. Parenteral administration may be by continuous infusion over a selected period of time.

A compound of the invention or a salt or solvate thereof may be orally administered, for example, with an inert diluent or with an assimilable edible carder, it may be enclosed in hard or soft shell gelatin capsules, it may be compressed into tablets, or it may be incorporated directly with the food of the diet. For oral therapeutic administration, the compound of the invention may be incorporated with excipient and used in the form of ingestible tablets, buccal tablets, troches, capsules, elixirs, suspensions, syrups, wafers, and the like.

A compound of the invention may also be administered parenterally or intraperitoneally. Solutions of a compound of the invention as a free base or pharmacologically acceptable salt or solvate can be prepared in water suitably mixed with a surfactant such as hydroxypropylcellulose. Dispersions can also be prepared in glycerol, liquid polyethylene glycols, DMSO, and mixtures thereof with or without alcohol, and in oils. Under ordinary conditions of storage and use, these preparations contain a preservative to prevent the growth of microorganisms. A person skilled in the art would know how to prepare suitable formulations. Conventional procedures and ingredients for the selection and preparation of suitable formulations are described, for example, in Remington's Pharmaceutical Sciences (1990—18th edition) and in The United States Pharmacopeia: The National Formulary (USP 24 NF19) published in 1999.

The pharmaceutical forms suitable for injectable use include sterile aqueous solutions or dispersion and sterile powders for the extemporaneous preparation of sterile injectable solutions or dispersions. In all cases, the form must be sterile and must be fluid to the extent that easy syringeability exists.

The compounds of the invention may be administered to an animal alone or in combination with pharmaceutically acceptable carriers, as noted above, the proportion of which is determined by the solubility and chemical nature of the compound, chosen route of administration and standard pharmaceutical practice.

The dosage of the compounds and/or compositions of the invention can vary depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the animal to be treated. One of skill in the art can determine the appropriate dosage based on the above factors. The compounds of the invention may be administered initially in a suitable dosage that may be adjusted as required, depending on the clinical response.

### EXAMPLES

### Reagents and Antibodies.

The anti-CD11b monoclonal antibody (mAb) 44a (IgG2a) [41] and the heterodimer-specific anti-CD18 mAb IB4 (IgG2a) [42, 43] were from ATCC. The mAb 24 (IgG1) [44] was from Abcam and the isotype control antibodies MOPC-21 (IgG1) and MOPC-173 (IgG2a), FITC-conjugated mAbs A85-1 (rat anti-mouse IgG1), R19-15 (rat anti-mouse IgG2a) and FITC-conjugated goat anti-mouse immunoglobulin were from BD Pharmingen (San Diego, Calif.). Rat anti-mouse GR1-FITC and Mac-1-PE were from BD Pharmingen (San Diego, Calif.). Human Fibrinogen (Plasminogen, vonWillebrand Factor and Fibronectin depleted) is from EnzymeResearch Laboratories (SouthBend, Ind.), bovine serum albumin (BSA) is from Sigma (St. Louis, Mich.). 384-well plates are from commercial sources (MaxiSorp from Nalgene (Rochester, N.Y.) and Highbind from Corning (Corning, N.Y.)). Non-fat milk is obtained from BioRad (Hercules, Calif.). Cell quantitation reagent MTS are from Promega (Madison, Wis.) and ATPLite from PerkinElmer (Boston, Mass.). Cell culture reagents are from Invitrogen Corp. (San Diego, Calif.) and Mediatech (Manassas, Va.). Fetal bovine serum is purchased from Atlanta Biologicals, Inc (Lawrenceville, Ga.).

### Cell Lines

K562 cells (ATCC) stably transfected with wild-type integrin CD11b/CD18 (K562 CD11b/CD18) have been described previously [33, 46]. K562 cells stably expressing CD11c/CD18 (K562 CD11c/CD18) were generated similarly. Mutant CD11bE320A has been described previously [47]. K562 cells stably transfected with mutant integrin CD11bE320A/CD18 (K562 E320A) were generated according to literature protocols [33, 46]. All cell lines were maintained in Iscove's Modified Dulbecco's Medium (IMDM) supplemented with 10% heat-inactivated fetal bovine serum, 50 IU/mL penicillin and streptomycin and 0.5 mg/mL G418.

### HTS Adhesion Assay

Cell adhesion assays with immobilized ligands were performed using the general protocol previously described and as illustrated in FIG. 1 [33]. Assays with all different K562 cell lines (K562, K562 CD11b/CD18, K562 CD11c/CD18 and K562 E320A) were performed in an identical fashion. Briefly, 384-well microplates (Corning 3700) were coated with 15 μg/mL fibrinogen (Fg) ligand in PBS⁺⁺ overnight at 4° C. At the same time, anti-CD18 (IB4) mAb was also coated. Subsequently, the coated wells were washed with 1% nonfat milk in TBS at room temperature for 1 hour. Next, the wells were washed 3 times with TBS, and the coated microplates were used in the HTS assays.

K562 CD11b/CD18, K562 CD11bE320A/CD18 and K562 CD11c/CD18 cells were washed with TBS, and cells were transferred to the Fg-coated wells of microplates in the assay buffer (Ca²⁺ and Mg²⁺ for physiological condition, Mn²⁺ as the Positive control). A 1 μL aliquot of the compounds were transferred to each washed well with multichannel pipet from the 384-well plates.

The assay plates were incubated at 37° C. in the presence of small-molecule compounds for 30 min. To dislodge the non-adherent cell, the assay plates were gently inverted and kept in inverted position for 20 min at room temperature.

The cells were fixed with 37% formaldehyde in a small volume and the plates were kept inverted for 1 h. Upon the fixation, the solutions in the well were discarded. Next, the cells were fluorescently labeled with DAPI (0.5 μM final in TBS with 0.1% Triton X-100) and the quantitated with “Opera High Content Screening System” (Perkin Elmer).

### Data Analysis:

Cell-adhesion under positive and negative control conditions was used to define the activity scale; signal corresponding to the number of non-small molecule treated cells adherent in activating buffer condition (in the presence of Mn²⁺ ions as agonist, PC) was considered maximum (100% binding) and the signal corresponding to the number of non-small molecule treated cells adherent in basal buffer condition (in the presence of 1 mM each of Ca²⁺ and Mg²⁺, NC) was considered minimum (0% binding).

$$\% Activation = \% Activation = \frac{( A - NC )}{( PC - NC )} * 100 \%$$

Compounds with EC50 (concentration needed for half-maximal increase in cell-adhesion) less than 8 were selected as Primary hits. The primary hits were cherry picked and retested in the assay with E320A Cells at the same concentration.

### Dose Response

The compounds were diluted in DMSO by the final concentration 10 μM, 5 μM, 2.5 μM, 1.25 μM and 0.625 μM and stocked as sealed in 384-well plates at −80° C.

| | Stock in Plates | 1 | 2 | 3 | 4 | 5 |
|---|---|---|---|---|---|---|
| STOCK | 10 mM | 1 mM | 0.5 mM | 0.25 mM | 0.125 mM | 0.0675 mM |
| Concentration | | | | | | |
| Buffer | | 10 | 5 | 2.5 | 1.25 | 0.675 |
| Concentration | | | | | | |

Compound Library: 92,500 compounds from the NIH's Molecular Libraries Probe Production Centers Network small molecule library. The compounds were cherry picked from the diluted compound plates used to generate the original primary screening data.

### Results

Primary screening of approximately 92,690 compounds was completed by Sanford-Burnham Medical Research Institute (SBMRI, San Diego, Calif.), which relies on the ability of small molecules to increase adhesion of mammalian K562 cells stably transfected with the wild type integrin CD11b/CD18 (K562 CD11b/CD18) to Fibrinogen, a physiologic ligand of integrin CD11b/CD18. K562 CD11b/CD18 cells showed virtually no binding to immobilized Fibrinogen (Fg) when incubated in the assay buffer (1 mM each of physiologic ions Ca²⁺ and Mg²⁺ in Tris buffered saline (TBS⁺⁺)) alone. Results were deposited at Pubchem (AID 1499).

We identified 845 compounds that showed ≥50% activity in the assay. These compounds were ordered from the NIH's small molecule repository for re-testing. We screened these compounds in-house using a 5-point dose-response series and binding of K562 CD11b/CD18 cells to Fibrinogen. We identified 258 compounds that produced dose-dependent increase in cell adhesion of K562 CD11b/CD18 cells (see Table 3).

To identify compounds that potentially target the CD11bA-domain, we used the K562 CD11bE320A/CD18 cells and performed a five point dose-response series. We also tested these compounds in assay with K562 CD11c/CD18 cells to find agonists of CD11c/CD18. We identified 82 compounds (see Table 4) that show high binding and thus were confirmed as agonist hits (hit confirmation rate of ˜53%). We did not find any overlap with the hits previously described in Bioassay AID 1499. The compounds listed in Table 1 are the same as those listed in Table 3, although the numbers are ordered sequentially in Table 1; likewise, the compounds listed in Table 2 are sequentially numbered, but are the same as those listed in Table 4.

TABLE 1
Beta2 Integrin Agonists
Pubchem Compound Identification Numbers

625239
629074
646977
650199
650604
650825
651007
652267
652695
655389
656131
658682
658936
658948
660042
660507
660662
660761
664182
664240
664564
665043
665142
665589
707842
743624
862194
960705
960708
984329
1011163
1016780
1017701
1045434
1076720
1078286
1132508
1135734
1144109
1164441
1193278
1212121
1219756
1226649
1227337
1245695
1254288
1255242
1255636
1262031
1272696
1290769
1295289
1308620
1309747
1310712
1314910
1317528
1322134
1331726
1334850
1345254
1347165
1352154
1356461
1358622
1398027
1418314
1442651
1494229
1554430
1554642
1564171
1599902
1745635
1764811
1773030
1825650
1827856
1844411
1871747
1891182
1943921
1949560
1951731
1964957
1967927
1973614
1976060
1984877
1993471
1997460
2023256
2030970
2030972
2064203
2067755
2078838
2081288
2083967
2098333
2108710
2121076
2126150
2132989
2144321
2145570
2145571
2150886
2163411
2182821
2193118
2208079
2214811
2252798
2254616
2313216
2321209
2327777
2338283
2365417
2366866
2369720
2408017
2421294
2422317
2448744
2462581
2537029
2549650
2566737
2566842
2569132
2766156
2829724
2831455
2836867
2912337
2941732
2958500
2965657
2997788
2998734
2998894
3000165
3101589
3160888
3162305
3197508
3207334
3226283
3235854
3237376
3237460
3237466
3238190
3238872
3241193
3241379
3241486
3242458
3244583
3244843
3244965
3272728
3287222
3352440
3395368
3466680
3908086
4168394
4325421
4412067
4483668
4831224
4835204
4896185
4971819
5143923
5212443
5287341
5307308
5307705
5308150
5309189
5309602
5309909
5334631
5334671
5342606
5343920
5346533
5347156
5349163
5350469
5423532
5431927
5494763
5497396
5516744
5521841
5624444
5683040
5727347
5736971
5739634
5769297
5774232
5801934
5912542
5950432
5971886
6012385
6030986
6079611
6098117
6098526
6291421
6299255
6402388
6508323
6513263
6879466
6880170
6880740
6881723
6892710
7109922
7191378
7206282
7285276
7424594
9550569
9551541
9551824
9557044
9564362
9568664
9594695
9617832
9619223
9774264
11840403
11948741
11957216
12004639
12004750
12005911
12005977
12006068
12006105
15944670
15944880
54680928

TABLE 2
Beta2 Integrin Agonists
Pubchem Compound Identification Numbers

629074
652267
652695
658682
658948
664182
665142
666410
960705
1017701
1078286
1132508
1144109
1164441
1227337
1255242
1255636
1262031
1295289
1308620
1310712
1317528
1331726
1334850
1356461
1494229
1554430
1554642
1599902
1745635
1825650
1891182
1943921
1949560
1951731
1964957
1967927
1984877
2030970
2030972
2083967
2144321
2150886
2163411
2214811
2252798
2254616
2321209
2365417
2422317
2569132
2831455
2997788
2998894
3207334
3235854
3237466
3241193
3242458
3244965
3466680
4483668
5143923
5308150
5309602
5342606
5346533
5431927
5494763
5521841
5736971
5769297
5912542
5971886
6098117
6299255
6880740
6892904
9568664
9774264
11957216
12004750
12005977
12006068

TABLE 3
Agonist Compounds Augmenting CD
11c/CD18 Binding to Fibrinogen

| Compound | EC₅₀ | EC₅₀ | EC₅₀ |
|---|---|---|---|
| ID No. | E320A | CD11c/CD18 | CD11b/CD18 |
| 1418314 | 9.57 | 3.444 | 4.487 |
| 5950432 | 9.83 | 6.487 | 5.891 |
| 9619223 | 35.71 | 11.8 | 6.181 |
| 658682 | 1.603 | 0.7531 | 0.6213 |
| 3241193 | 1.281 | 1.256 | 0.6911 |
| 665589 | 14.55 | 5.108 | 5.962 |
| 2997788 | 5.269 | 2.684 | 4.718 |
| 1984877 | 3.071 | 2.537 | 2.16 |
| 9564362 | 12.22 | 9.956 | 4.418 |
| 1345254 | 7.527 | 4.504 | 1.223 |
| 2327777 | 21.86 | Infinite | 2.567 |
| 2965657 | 12.77 | Infinite | 0.7002 |
| 656131 | 9.971 | 9.592 | 6.78 |
| 651007 | 8.178 | 4.878 | 6.586 |
| 2078838 | 72.4 | 10.8 | 6.682 |
| 2829724 | 18.28 | 11.43 | 7.444 |
| 4412067 | 11.41 | 9.968 | 6.136 |
| 4168394 | 8.854 | 9.336 | 6.401 |
| 625239 | 7.743 | 4.334 | 6.488 |
| 2836867 | 121 | 11.59 | 8.702 |
| 9557044 | 10.49 | 10.11 | 6.587 |
| 5309602 | 6.416 | 4.782 | 2.56 |
| 1764811 | 19.31 | 9.827 | 9.505 |
| 1254288 | 11.51 | 10.35 | 8.803 |
| 1219756 | 9.943 | 14.1 | 4.02 |
| 6098117 | 3.761 | 2.54 | 1.527 |
| 2030970 | Very Low | 9.608 | Very Low |
| 3244583 | 9.704 | 10.45 | 5.562 |
| 1322134 | 12.12 | 9.676 | 4.309 |
| 2462581 | 12.58 | 9.695 | 4.252 |
| 1825650 | 6.615 | 9.55 | 4.969 |
| 2448744 | 21.8 | 8.596 | 4.546 |
| 3237376 | 12.23 | 10.09 | 7.32 |
| 9551541 | 8.228 | 6.16 | 2.612 |
| 1226649 | 8.47 | 5.653 | 3.605 |
| 1255242 | 3.409 | 5.028 | 2.569 |
| 11957216 | 6.755 | 5.077 | 4.312 |
| 3352440 | 10.1 | 7.662 | 3.078 |
| 2064203 | 8.968 | 5.637 | 5.308 |
| 2150886 | 5.052 | 5.168 | 2.129 |
| 1356461 | 4.023 | 1.428 | 2.267 |
| 3226283 | 6.695 | 6.708 | 3.767 |
| 660042 | 9.35 | 9.649 | 6.887 |
| 1871747 | 15.44 | 7.236 | 3.759 |
| 7285276 | 16.3 | 12.29 | 3.374 |
| 3241486 | 9.083 | 9.15 | 7.47 |
| 2549650 | 10.33 | 13.19 | 4.32 |
| 629074 | 5.158 | 5.803 | 2.357 |
| 2912337 | 12.41 | 11547 | 3.984 |
| 15944670 | 21.4 | Infinite | 4.866 |
| 9550569 | 8.217 | 5.181 | 8.358 |
| 6299255 | 3.618 | 1.305 | 3.228 |
| 650825 | 9.861 | 8.93 | 2.84 |
| 2566737 | 8.42 | 7.78 | 5.658 |
| 1964957 | 6.467 | 5.924 | 10.55 |
| 3242458 | 2.828 | 1.558 | 1.886 |
| 1599902 | 5.709 | 576.8 | 2.23 |
| 1144109 | 7.059 | 2.348 | 3.059 |
| 1227337 | 4.191 | 3.999 | 3.317 |
| 2365417 | 2.826 | 5.977 | 4.042 |
| 743624 | 15.67 | 8.287 | 4.743 |
| 1773030 | 7.744 | 3.516 | 3.516 |
| 1078286 | 6.921 | 6.917 | 2.501 |
| 3237466 | 3.689 | 3.759 | 1.352 |
| 1255636 | 0.6782 | 19.01 | Very Low |
| 5624444 | 15.29 | 3.722 | 7.64 |
| 1997460 | 6.227 | 1.635 | 1.866 |
| 1262031 | 1.603 | 7.208 | 7.187 |
| 1193278 | 5.166 | 1.268 | 2.77 |
| 3237460 | 135.3 | 9.273 | |
| 2408017 | 2.02 × 10⁸ | 4.739 | 5.803 |
| 2098333 | 0.4410 (reverse) | 5.943 | 6.841 |
| 2338283 | Infinite | 4.976 | 7.786 |
| 1317528 | 3.623 | 2.36 | 2.135 |
| 7424594 | 10.26 | 3.646 | 4.064 |
| 2081288 | 12.68 | 5.523 | 6.288 |
| 1844411 | 7.039 | 2.319 | 6.161 |
| 2067755 | 78.26 | 4.061 | 4.392 |
| 6402388 | 11.24 | 3.923 | 4.805 |
| 2145570 | 8.248 | 3.054 | 2.799 |
| 1310712 | 2.395 | 1.993 | 1.286 |
| 1398027 | 18.21 | 5.482 | 5.138 |
| 665142 | 4.917 | 1.204 | 2.625 |
| 3908086 | 12.17 | 4.359 | 4.435 |
| 6513263 | 13.91 | 3.332 | 3.424 |
| 1993471 | 163.7 | 4.377 | 3.072 |
| 4483668 | 5.421 | 5.253 | 4.636 |
| 1967927 | 2.67 | 2.738 | 2.488 |
| 5683040 | 14.44 | 5.262 | 6.596 |
| 650199 | 13.35 | 4.982 | 6.354 |
| 5769297 | 2.527 | 2.125 | 2.535 |
| 6879466 | 170.8 | 5.599 | 7.095 |
| 984329 | 9.234 | 5.252 | 8.206 |
| 3207334 | 5.171 | 3.81 | 1.99 |
| 655389 | 8.489 | 4.68 | 6.707 |
| 2126150 | Infinite | 4.941 | 8.151 |
| 1272696 | Infinite | 8.433 | 4.131 |
| 1314910 | 11.74 | 9.413 | 4.318 |
| 1290769 | 7.817 | 3.676 | 5.552 |
| 1358622 | 13.67 | 2.725 | 2.874 |
| 12004639 | 10.24 | 3.412 | 5.578 |
| 2421294 | 9.807 | 5.044 | 4.578 |
| 5727347 | 10.24 | 2.609 | 5.935 |
| 2254616 | 4.425 | 1.31 | 1.869 |
| 2941732 | 35.59 | 7.858 | 2.149 |
| 3238190 | 10.5 | 5.961 | 7.597 |
| 3162305 | 10.39 | 4.798 | 9.03 |
| 3101589 | 13.45 | 5.146 | 6.78 |
| 1331726 | 2.459 | 0.86 | 2.644 |
| 12006068 | 6.837 | 2.59 | 2.463 |
| 4831224 | 11.15 | 4.126 | 6.618 |
| 2252798 | 4.095 | 2.661 | 4.063 |
| 2321209 | 5.298 | 2.646 | 2.63 |
| 2132989 | 18 | 14.89 | 6.378 |
| 11948741 | 8.089 | 4.807 | 3.879 |
| 3197508 | 8.477 | 8.933 | 6.03 |
| 1334850 | 2.695 | 1.014 | 1.379 |
| 1554642 | 7.9 | 3.947 | 5.26 |
| 1308620 | 3.303 | 4.283 | 6.26 |
| 5774232 | 8.841 | 10.51 | 7.721 |
| 12004750 | 6.234 | 5.897 | 5.841 |
| 664182 | 4.238 | 3.959 | 3.781 |
| 6880740 | 4.985 | 4.421 | 2.599 |
| 5343920 | Very Low | 3.935 | 5.408 |
| 12005911 | 12.12 | 10.27 | 11.21 |
| 660507 | 18.5 | 9.944 | 7.06 |
| 1494229 | 6.529 | 2.563 | 6.717 |
| 1949560 | 5.463 | 2.686 | 5.531 |
| 1164441 | 4.004 | 2.51 | 2.775 |
| 1745635 | 3.706 | Very low | 70.37 |
| 1076720 | 14.86 | 5.749 | 4.686 |
| 5309909 | 258.7 | Infinite | 5.514 |
| 1943921 | 4.122 | 1.368 | 3.5 |
| 2569132 | 6.292 | 3.675 | 5.281 |
| 2208079 | 567.9 | 1.404 × 10⁶ | 3.472 |
| 1212121 | 18.84 | 0.9866 | 7.579 |
| 3000165 | 9.018 | 5.13 | 7.913 |
| 6012385 | 25.09 | 158.6 | 5.699 |
| 6098526 | 13.36 | 9.173 | 5.415 |
| 2366866 | 8.59 | 3.036 | 7.267 |
| 7109922 | 8.761 | 1.617 | 4.856 |
| 2182821 | 8.515 | 4.964 | 5.36 |
| 5307705 | 9.933 | 10.18 | 3.421 |
| 1011163 | 12.91 | 4.091 | 5.958 |
| 3238872 | 19.11 | 4.396 | 6.336 |
| 1554430 | 4.68 | 5.212 | 1.301 |
| 9774264 | 4.164 | 9.43 | 1.034 |
| 2083967 | 3.331 | 2.23 | 2.696 |
| 2958500 | 8.939 | 4.458 | 7.468 |
| 650604 | 9.43 | 7.155 | 7.485 |
| 1045434 | 9.84 | Infinite | 3.981 |
| 658948 | 0.9942 | 0.7315 | 0.5641 |
| 1309747 | 10.49 | 11.94 | 4.47 |
| 7206282 | 10.74 | 7.291 | 6.335 |
| 15944880 | 10.12 | 5.883 | 4.253 |
| 1017701 | 8.108 | 2.5 | 2.715 |
| 5287341 | 8.975 | 4.84 | 4.158 |
| 5347156 | 13.67 | 7.01 | 6.211 |
| 5307308 | 18.7 | 4.443 | 3.528 |
| 1951731 | 3.654 | 2.045 | 4.207 |
| 6030986 | 8.101 | 8.539 | 6.117 |
| 3244965 | 4.911 | 3.462 | 4.028 |
| 5736971 | 6.547 | 4.599 | 4.499 |
| 1827856 | 7.034 | 4.26 | 6.326 |
| 5516744 | 13.56 | 9.19 | 6.956 |
| 2030972 | 6.689 | Very Low | Very Low |
| 5350469 | 14.36 | 9.728 | 6.896 |
| 1976060 | 0.3153 (Reverse) | 1.86 | 1.904 |
| 12005977 | 6.715 | 9.39 | 5.047 |
| 9568664 | 6.192 | 2.988 | 3.728 |
| 5971886 | 4.657 | 1.201 | 1.102 |
| 2831455 | 5.084 | 4.12 | 6.866 |
| 660662 | 9.733 | 8.811 | 7.714 |
| 1245695 | 8.466 | 4.8 | 6.953 |
| 6881723 | 7.326 | 4.849 | 5.939 |
| 658936 | 0.2092 (Reverse) | 1.669 (Reverse) | 1.164 |
| 5494763 | 4.902 | 4.717 | 4.955 |
| 1442651 | 10.78 | 7.064 | 6.796 |
| 1973614 | 43.61 | 3.671 | 4.648 |
| 1132508 | 4.443 | 0.76 | 5.39 |
| 960705 | 3.492 | 1.398 | 2.281 |
| 9594695 | 17.52 | 5.172 | 7.539 |
| 5349163 | 10.92 | 7.869 | 5.733 |
| 5497396 | 11.66 | 60.29 | 7.956 |
| 7191378 | 8.133 | 52.49 | 5.216 |
| 5423532 | 8.978 | 8.495 | 6.044 |
| 2537029 | 8758 | 9.463 | 7.907 |
| 6079611 | 9.051 | 8.423 | 3.176 |
| 3235854 | 5.555 | 4.021 | 2.705 |
| 2766156 | 10.66 | 5.855 | 6.556 |
| 652695 | 4.859 | 2.62 | 4.615 |
| 3272728 | 10.8 | 4.58 | 7.433 |
| 5212443 | 12.98 | 6.611 | 7.301 |
| 5346533 | 5.807 | 5.117 | 3.459 |
| 1295289 | 3.619 | 1.414 | 2.67 |
| 5143923 | 5.315 | 4.986 | 3.014 |
| 3466680 | 5.991 | 5.433 | 6.257 |
| 6508323 | 12.63 | 4.033 | 6.3 |
| 1891182 | 6.863 | 3.882 | 5.267 |
| 5334631 | 12.95 | 6.826 | 5.391 |
| 1347165 | 171.3 | 8.482 | 4.597 |
| 960708 | 101.3 | 9.435 | 6.816 |
| 2566842 | 7.571 | 4.903 | 5.075 |
| 2214811 | 4.678 | 2.448 | 2.536 |
| 2108710 | 62.49 | 9.811 | 3.856 |
| 652267 | 6.001 | 3.384 | 5.009 |
| 5912542 | 6.981 | 4.747 | 7.767 |
| 4835204 | Infinite | 20.24 | 6.44 |
| 862194 | 15.19 | 8.549 | 3.753 |
| 5308150 | 1.261 | Very Low | 17.34 |
| 3160888 | 11.43 | 9.383 | 5.432 |
| 3395368 | 8.783 | 5.249 | 4.844 |
| 2369720 | 11.25 | 5.501 | 3.597 |
| 664564 | 20.39 | 9.685 | 7.03 |
| 4896185 | 11.84 | 9.714 | 4.463 |
| 3287222 | 16.49 | 7.234 | 3.752 |
| 2145571 | 12.09 | 5.125 | 4.735 |
| 6892710 | 12.44 | 4.062 | 3.363 |
| 2193118 | 38.17 | 33.96 | 2.924 |
| 6880170 | 11.28 | 5.051 | 5.095 |
| 11840403 | 13.36 | 34.74 | 1.912 |
| 2998734 | 8.015 | 2.989 | 2.837 |
| 2121076 | 19.37 | 6.383 | 5.048 |
| 2422317 | 5.703 | 2.588 | 3.35 |
| 2023256 | 26.06 | 1544 | 6.201 |
| 5342606 | 5.159 | 2.603 | 2.942 |
| 5521841 | 3.625 | 2.685 | 2.512 |
| 665043 | 9.234 | 2.475 | 4.072 |
| 2313216 | 28.35 | 16.32 | 5.865 |
| 646977 | 10.1 | 7.248 | 5.644 |
| 2144321 | 6.486 | 5.863 | 5.067 |
| 1135734 | 9.418 | 4.67 | 2.074 |
| 4971819 | 60.19 | 9.326 | 5.255 |
| 9551824 | 22.3 | 7.315 | 6.672 |
| 5739634 | 7.099 | 3.211 | 1.632 |
| 660761 | 8.803 | 5.499 | 5.625 |
| 1016780 | 7.818 | 5.271 | 4.561 |
| 1564171 | 14.83 | 7.555 | 5.828 |
| 2998894 | 5.857 | 2.646 | 3.053 |
| 12006105 | 9.854 | 4.737 | 4.54 |
| 5334671 | 16.92 | 7.64 | 7.91 |
| 5309189 | 14.24 | 10.98 | 4.852 |
| 1352154 | 8.01 | 4.123 | 1.262 |
| 2163411 | 6.56 | 7.497 | 1.77 |
| 5801934 | 7.85 | 2.848 | 2.697 |
| 664240 | 13.78 | 5.235 | 4.596 |
| 6291421 | 12.48 | 6.923 | 1.582 |
| 3241379 | 12.62 | 6.826 | 6.934 |
| 3244843 | 46.2 | 7.766 | 3.591 |
| 54680928 | 16.09 | 4.623 | 4.759 |
| 4325421 | 999.2 | 11.79 | 7.49 |
| 9617832 | 11.72 | 5.827 | 4.411 |
| 707842 | 14.99 | Infinite | 6.463 |
| 5431927 | 2.355 | 1.751 | 2.008 |

TABLE 4
Agonistic Compounds Targeting the CD11bA-Domain

| Compound | EC₅₀ | EC₅₀ | EC₅₀ |
|---|---|---|---|
| ID No. | E320A | CD11c/CD18 | CD11b/CD18 |
| 1255636 | 0.6782 | N/A Very Low | N/A Very Low |
| 658948 | 0.9942 | 0.5641 | 0.7315 |
| 5308150 | 1.261 | N/A Very Low | N/A Very Low |
| 3241193 | 1.281 | 0.6911 | 1.256 |
| 658682 | 1.603 | 0.6213 | 0.7531 |
| 5431927 | 2.355 | 2.008 | 1.751 |
| 1310712 | 2.395 | 1.286 | 1.993 |
| 1331726 | 2.459 | 2.644 | 0.8603 |
| 5769297 | 2.527 | 2.535 | 2.125 |
| 1967927 | 2.67 | 2.488 | 2.738 |
| 1334850 | 2.695 | 1.379 | 1.014 |
| 2365417 | 2.826 | 4.042 | 5.977 |
| 3242458 | 2.828 | 1.886 | 1.558 |
| 1984877 | 3.071 | 2.16 | 2.537 |
| 1308620 | 3.303 | 6.268 | 4.283 |
| 2083967 | 3.331 | 2.696 | 2.23 |
| 1255242 | 3.409 | 2.569 | 5.028 |
| 960705 | 3.492 | 2.281 | 1.398 |
| 6299255 | 3.618 | 3.228 | 1.305 |
| 1295289 | 3.619 | 2.67 | 1.414 |
| 1317528 | 3.623 | 2.135 | 2.63 |
| 5521841 | 3.625 | 2.512 | 2.685 |
| 1951731 | 3.654 | 4.207 | 2.045 |
| 3237466 | 3.689 | 1.352 | 3.759 |
| 1745635 | 3.706 | N/A Very Low | N/A Very Low |
| 6098117 | 3.761 | 1.527 | 2.54 |
| 1164441 | 4.004 | 2.775 | 2.51 |
| 1356461 | 4.023 | 2.267 | 1.428 |
| 2252798 | 4.095 | 4.063 | 2.661 |
| 1943921 | 4.122 | 3.5 | 1.368 |
| 9774264 | 4.164 | 1.034 | 9.43 |
| 1227337 | 4.191 | 3.317 | 3.999 |
| 664182 | 4.238 | 3.781 | 3.959 |
| 2254616 | 4.425 | 1.869 | 1.31 |
| 1132508 | 4.443 | 5.39 | 0.7609 |
| 5971886 | 4.657 | 1.102 | 1.201 |
| 2214811 | 4.678 | 2.536 | 2.448 |
| 1554430 | 4.68 | 1.301 | 5.212 |
| 652695 | 4.859 | 4.615 | 2.62 |
| 5494763 | 4.902 | 4.955 | 4.717 |
| 3244965 | 4.911 | 4.028 | 3.462 |
| 665142 | 4.917 | 2.625 | 1.204 |
| 6880740 | 4.985 | 2.599 | 4.421 |
| 2150886 | 5.052 | 2.129 | 5.168 |
| 2831455 | 5.084 | 6.866 | 4.12 |
| 629074 | 5.158 | 2.357 | 5.803 |
| 5342606 | 5.159 | 2.942 | 2.603 |
| 3207334 | 5.171 | 1.99 | 3.81 |
| 2997788 | 5.269 | 4.718 | 2.684 |
| 2321209 | 5.298 | 2.63 | 2.646 |
| 5143923 | 5.315 | 3.014 | 4.986 |
| 4483668 | 5.421 | 4.636 | 5.253 |
| 1949560 | 5.463 | 5.531 | 2.686 |
| 6892904 | 5.486 | 2.416 | 2.063 |
| 3235854 | 5.555 | 2.705 | 4.021 |
| 2422317 | 5.703 | 3.35 | 2.588 |
| 1599902 | 5.709 | 2.23 | 576.8 |
| 5346533 | 5.807 | 3.459 | 5.117 |
| 2998894 | 5.857 | 3.053 | 2.646 |
| 3466680 | 5.991 | 6.257 | 5.433 |
| 652267 | 6.001 | 5.009 | 3.384 |
| 9568664 | 6.192 | 3.728 | 2.988 |
| 666410 | 6.217 | 2.453 | 2.949 |
| 12004750 | 6.234 | 5.841 | 5.897 |
| 2569132 | 6.292 | 5.281 | 3.675 |
| 5309602 | 6.416 | 2.56 | 4.782 |
| 1964957 | 6.467 | 10.55 | 5.924 |
| 2144321 | 6.486 | 5.067 | 5.863 |
| 1494229 | 6.529 | 6.717 | 2.563 |
| 5736971 | 6.547 | 4.499 | 4.599 |
| 2163411 | 6.56 | 1.77 | 7.497 |
| 1825650 | 6.615 | 4.969 | 9.55 |
| 2030972 | 6.689 | N/A Very Low | N/A Very Low |
| 12005977 | 6.715 | 5.047 | 9.93 |
| 11957216 | 6.755 | 4.312 | 5.077 |
| 12006068 | 6.837 | 2.463 | 2.59 |
| 1891182 | 6.863 | 5.267 | 3.882 |
| 1078286 | 6.921 | 2.501 | 6.917 |
| 5912542 | 6.981 | 7.767 | 4.747 |
| 1144109 | 7.059 | 3.059 | 2.348 |
| 1554642 | 7.9 | 5.26 | 3.947 |
| 1017701 | 8.108 | 2.715 | 2.5 |
| 2030970 | 9.991 | N/A Very Low | N/A Very Low |
| 1262031 | 1.603 | 7.208 | 7.187 |

All patents and patent applications are herein incorporated by reference in their entirety to the same extent as if each individual patent or patent application was specifically and individually indicated to be incorporated by reference in its entirety.

Those skilled in the art will recognize, or be able to ascertain using no more than routine experimentation, many equivalents to the specific embodiments of the invention described herein. Such equivalents are intended to be encompassed by the following claims.

### REFERENCES

1. Mayadas, T. N. and X. Cullere, Neutrophil beta2 integrins: moderators of life or death decisions. Trends Immunol, 2005. 26(7): p. 388-95.
2. Ley, K., et al., Getting to the site of inflammation: the leukocyte adhesion cascade updated. Nat Rev Immunol, 2007. 7(9): p. 678-89.
3. Hynes, R. O., Integrins: bidirectional, allosteric signaling machines. Cell, 2002. 110(6): p. 673-87.
4. Amaout, M. A., Leukocyte adhesion molecules deficiency: its structural basis, pathophysiology and implications for modulating the inflammatory response. Immunol Rev, 1990. 114: p. 145-80.
5. Simon, D. I., et al., Decreased neointimal formation in Mac-1 (−/−) mice reveals a role for inflammation in vascular repair after angioplasty. J Clin Invest, 2000. 105(3): p. 293-300.
6. Cao, C., et al., A specific role of integrin Mac-1 in accelerated macrophage efflux to the lymphatics. Blood, 2005. 106(9): p. 3234-41.
7. Tang, T., et al., A role for Mac-1 (CD11b/CD18) in immune complex-stimulated neutrophil function in vivo: Mac-1 deficiency abrogates sustained Fcgamma receptor-dependent neutrophil adhesion and complement-dependent proteinuria in acute glomerulonephritis. J Exp Med, 1997. 186(11): p. 1853-63.
8. Plow, E. F., et al., Ligand binding to integrins. J Biol Chem, 2000. 275(29): p. 21785-8.
9. Soriano, S. G., et al., Mice deficient in Mac-1 (CD11b/CD18) are less susceptible to cerebral ischemia/reperfusion injury. Stroke, 1999. 30(1): p. 134-9. 10. Kubota, Y., et al., M-CSF inhibition selectively targets pathological angiogenesis and lymphangiogenesis. J Exp Med, 2009. 206(5): p. 1089-102.
11. Ahn, G. O., et al., Inhibition of Mac-1 (CD11b/CD18) enhances tumor response to radiation by reducing myeloid cell recruitment. Proc Natl Acad Sci USA. 107(18): p. 8363-8.
12. Ophascharoensuk, V., et al., Role of intrinsic renal cells versus infiltrating cells in glomerular crescent formation. Kidney Int, 1998. 54(2): p. 416-25.
13. Le Hir, M., et al., Podocyte bridges between the tuft and Bowman's capsule: an early event in experimental crescentic glomerulonephritis. J Am Soc Nephrol, 2001. 12(10): p. 2060-71.
14. Tang, T., et al., A role for Mac-1 (CD11b/CD18) in immune complex-stimulated neutrophil function in vivo: Mac-1 deficiency abrogates sustained Fcgamma receptor-dependent neutrophil adhesion and complement-dependent proteinuria in acute glomerulonephritis. J Exp Med, 1997. 186(11): p. 1853-63.
15. Fan, S. T. and T. S. Edgington, Coupling of the adhesive receptor CD11b/CD18 to functional enhancement of effector macrophage tissue factor response. J Clin Invest, 1991. 87(1): p. 50-7.
16. Whitlock, B. B., et al., Differential roles for alpha(M)beta(2) integrin clustering or activation in the control of apoptosis via regulation of akt and ERK survival mechanisms. Cell Biol, 2000. 151(6): p. 1305-20.
17. Rezzonico, R., et al., Ligation of CD11b and CD11c beta(2) integrins by antibodies or soluble CD23 induces macrophage inflammatory protein 1 alpha (MIP-1 alpha) and MIP-1 beta production in primary human monocytes through a pathway dependent on nuclear factor-kappaB. Blood, 2001. 97(10): p. 2932-40.
18. Guha, M. and N. Mackman, The phosphatidylinositol 3-kinase-Akt pathway limits lipopolysaccharide activation of signaling pathways and expression of inflammatory mediators in human monocytic cells. J Biol Chem, 2002. 277(35): p. 32124-32.
19. Rubel, C., et al., Fibrinogen-CD11b/CD18 interaction activates the NF-kappa B pathway and delays apoptosis in human neutrophils. Eur J Immunol, 2003. 33(5): p. 1429-38.
20. Kettritz, R., et al., Integrins and cytokines activate nuclear transcription factor-kappaB in human neutrophils. J Biol Chem, 2004. 279(4): p. 2657-65.
21. Giancotti, F. G. and E. Ruoslahti, Integrin signaling. Science, 1999. 285(5430): p. 1028-32.
22. Jaeschke, H., et al., Functional inactivation of neutrophils with a Mac-1 (CD11b/CD18) monoclonal antibody protects against ischemia-reperfusion injury in rat liver. Hepatology, 1993. 17(5): p. 915-23.
23. Rogers, C., E. R. Edelman, and D. I. Simon, A mAb to the beta2-leukocyte integrin Mac-1 (CD11b/CD18) reduces intimal thickening after angioplasty or stent implantation in rabbits. Proc Natl Acad Sci USA, 1998. 95(17): p. 10134-9.
24. Wilson, I., et al., Inhibition of neutrophil adherence improves postischemic ventricular performance of the neonatal heart. Circulation, 1993. 88(5 Pt 2): p. 11372-9.
25. Plow, E. F. and L. Zhang, A MAC-1 attack: integrin functions directly challenged in knockout mice. J Clin Invest, 1997. 99(6): p. 1145-6.
26. Yonekawa, K. and J. M. Harlan, Targeting leukocyte integrins in human diseases. J Leukoc Biol, 2005. 77(2): p. 129-40.
27. Hu, X., et al., beta2-integrins in demyelinating disease: not adhering to the paradigm. J Leukoc Biol, 2010. 87(3): p. 397-403.
28. Dove, A., CD18 trials disappoint again. Nat Biotechnol, 2000. 18(8): p. 817-8.
29. Shimizu, K., et al., Leukocyte integrin Mac-1 promotes acute cardiac allograft rejection. Circulation, 2008. 117(15): p. 1997-2008.
30. Ramamoorthy, C., et al., CD18 adhesion blockade decreases bacterial clearance and neutrophil recruitment after intrapulmonary E. coli, but not after S. aureus. J Leukoc Biol, 1997. 61(2): p. 167-72.
31. Lum, A. F., et al., Dynamic regulation of LFA-1 activation and neutrophil arrest on intercellular adhesion molecule 1 (ICAM-1) in shear flow. J Biol Chem, 2002. 277(23): p. 20660-70.
32. Allison, M., PML problems loom for Rituxan. Nat. Biotechnol, 2010. 28(2): p. 105-6.
33. Park, J. Y., M. A. Arnaout, and V. Gupta, A simple, no-wash cell adhesion-based high-throughput assay for the discovery of small-molecule regulators of the integrin CD11b/CD18. J Biomol Screen, 2007. 12(3): p. 406-17.
34. Faridi, M. H., et al., Identification of novel agonists of the integrin CD11b/CD18. Bioorg Med Chem Lett, 2009. 19(24): p. 6902-6.
35. Kuijpers, T. W., et al., Freezing adhesion molecules in a state of high-avidity binding blocks eosinophil migration. J Exp Med, 1993. 178(1): p. 279-84.
36. Semmrich, M., et al., Importance of integrin LFA-1 deactivation for the generation of immune responses. J Exp Med, 2005. 201(12): p. 1987-98.
37. Park, E. J., et al., Distinct roles for LFA-1 affinity regulation during T-cell adhesion, diapedesis, and interstitial migration in lymph nodes. Blood, 2010. 115(8): p. 1572-81.
38. Park, E. J., et al., Aberrant activation of integrin alpha4beta7 suppresses lymphocyte migration to the gut. J Clin Invest, 2007. 117(9): p. 2526-38.
39. Faridi, M. H., et al., Identification of novel agonists of the integrin CD11b/CD18. Bioorg Med Chem Lett, 2009.
40. Wang, Y., et al., Leukocyte engagement of platelet glycoprotein Ibalpha via the integrin Mac-1 is critical for the biological response to vascular injury. Circulation, 2005. 112(19): p. 2993-3000.
41. Arnaout, M. A., et al., Inhibition of phagocytosis of complement C3- or immunoglobulin G-coated particles and of C3bi binding by monoclonal antibodies to a monocyte-granulocyte membrane glycoprotein (Mol). J Clin Invest, 1983. 72(1): p. 171-9.
42. Wright, S. D., et al., Identification of the C3bi receptor of human monocytes and macrophages by using monoclonal antibodies. Proc Natl Acad Sci USA, 1983. 80(18): p. 5699-703.
43. Hogg, N., et al., A novel leukocyte adhesion deficiency caused by expressed but nonfunctional beta2 integrins Mac-1 and LFA-1. J Clin Invest, 1999. 103(1): p. 97-106.
44. Dransfield, I. and N. Hogg, Regulated expression of Mg2+ binding epitope on leukocyte integrin alpha subunits. EMBO J, 1989. 8(12): p. 3759-65.
45. Coxon, A., et al., A novel role for the beta 2 integrin CD11b/CD18 in neutrophil apoptosis: a homeostatic mechanism in inflammation. Immunity, 1996. 5(6): p. 653-66.
46. Gupta, V., et al., The beta-tail domain (betaTD) regulates physiologic ligand binding to integrin CD11b/CD18. Blood, 2007. 109(8): p. 3513-20.
47. Alonso, J. L., et al., Does the integrin alphaA domain act as a ligand for its betaA domain? Curr Biol, 2002. 12(10): p. R340-2.
48. Gupta, V., HTS identification of compounds that enhance the binding of CD11b/CD18 to fibrinogen via a luminescence assay. Pubchem Assay ID 1499, 2009.
49. Chen, L. Y., et al., Impaired glucose homeostasis, neutrophil trafficking and function in mice lacking the glucose-6-phosphate transporter. Hum Mol Genet, 2003. 12(19): p. 2547-58.
50. Bergmeier, W., et al., Mice lacking the signaling molecule CaIDAG-GEFI represent a model for leukocyte adhesion deficiency type III. J Clin Invest, 2007. 117(6): p. 1699-707.
51. Szczur, K., et al., Rho GTPase CDC42 regulates directionality and random movement via distinct MAPK pathways in neutrophils. Blood, 2006. 108(13): p. 4205-13.
52. Zigmond, S. H., Orientation chamber in chemotaxis. Methods Enzymol, 1988. 162: p. 65-72.
53. Pluskota, E., et al., Neutrophil apoptosis: selective regulation by different ligands of integrin alphaMbeta2. J Immunol, 2008. 181(5): p. 3609-19.
54. Li, R. and M. A. Amaout, Functional analysis of the beta 2 integrins. Methods Mol Biol, 1999. 129: p. 105-24.
55. Xiong, J. P., et al., An isoleucine-based allosteric switch controls affinity and shape shifting in integrin CD11b A-domain. J Biol Chem, 2000. 275(49): p. 38762-7.
56. Lu, C., et al., Epitope mapping of antibodies to the C-terminal region of the integrin beta 2 subunit reveals regions that become exposed upon receptor activation. J Immunol, 2001. 166(9): p. 5629-37.
57. Xiong, J. P., et al., New insights into the structural basis of integrin activation. Blood, 2003. 102(4): p. 1155-9.
58. Gabeler, E. E., et al., A comparison of balloon injury models of endovascular lesions in rat arteries. BMC Cardiovasc Disord, 2002. 2: p. 16.
59. Renshaw, S. A., et al., A transgenic zebrafish model of neutrophilic inflammation. Blood, 2006. 108(13): p. 3976-8.
60. Nusslein-Volhard, C., Zebrafish—A Practical Approach. Practical Approach Series. 2002: Oxford University Press.
61. Lee, J. O., et al., Crystal structure of the A domain from the alpha subunit of integrin CR3 (CD11b/CD18). Cell, 1995. 80(4): p. 631-8.
62. Xiong, J. P., et al., An isoleucine-based allosteric switch controls affinity and shape shifting in integrin CD11b A-domain. J Biol Chem, 2000. 275(49): p. 38762-7.
63. Weitz-Schmidt, G., et al., Statins selectively inhibit leukocyte function antigen-1 by binding to a novel regulatory integrin site. Nat Med, 2001. 7(6): p. 687-92.
64. Lee, J. O., et al., Two conformations of the integrin A-domain (I-domain): a pathway for activation? Structure, 1995. 3(12): p. 1333-40.
65. Shimaoka, M., et al., Stabilizing the integrin alpha M inserted domain in alternative conformations with a range of engineered disulfide bonds. Proc Natl Acad Sci USA, 2002. 99(26): p. 16737-41.
66. McCleverty, C. J. and R. C. Liddington, Engineered allosteric mutants of the integrin alphaMbeta2 I domain: structural and functional studies. Biochem J, 2003. 372(Pt 1): p. 121-7.
67. Sherman, W., et al., Novel procedure for modeling ligand/receptor induced fit effects. J Med Chem, 2006. 49(2): p. 534-53.
68. Kevin, J. B., et al., Scalable Algorithms for Molecular Dynamics Simulations on Commodity Clusters. Proceedings of the ACM/IEEE Conference on Supercomputing (SC06), Tampa, Fla., Nov. 11-17, 2006.
69. Barnard, J. W., et al., Neutrophil inhibitory factor prevents neutrophil-dependent lung injury. J Immunol, 1995. 155(10): p. 4876-81.
70. Zerria, K., et al., Recombinant integrin CD11b A-domain blocks polymorphonuclear cells recruitment and protects against skeletal muscle inflammatory injury in the rat. Immunology, 2006. 119(4): p. 431-40.
71. Feng, Y., et al., Peptides derived from the complementarity-determining regions of anti-Mac-1 antibodies block intercellular adhesion molecule-1 interaction with Mac-1. J Biol Chem, 1998. 273(10): p. 5625-30.
72. Li, R., et al., Two functional states of the CD11b A-domain: correlations with key features of two Mn2+-complexed crystal structures. J Cell Biol, 1998. 143(6): p. 1523-34.
73. Abad-Zapatero, C. and J. T. Metz, Ligand efficiency indices as guideposts for drug discovery. Drug Discov Today, 2005. 10(7): p. 464-9.
74. Friesner, R. A., et al., Glide: a new approach for rapid, accurate docking and scoring. 1. Method and assessment of docking accuracy. J Med Chem, 2004. 47(7): p. 1739-49.
75. Guimaraes, C. R. and M. Cardozo, MM-GB/SA rescoring of docking poses in structure-based lead optimization. J Chem Inf Model, 2008. 48(5): p. 958-70.
76. Bjorklund, M., et al., Stabilization of the activated alphaMbeta2 integrin by a small molecule inhibits leukocyte migration and recruitment. Biochemistry, 2006. 45(9): p. 2862-71.
77. Liu, L., et al., Requirement for RhoA kinase activation in leukocyte de-adhesion. J Immunol, 2002. 169(5): p. 2330-6.
78. Huttenlocher, A., M. H. Ginsberg, and A. F. Horwitz, Modulation of cell migration by integrin-mediated cytoskeletal linkages and ligand-binding affinity. J Cell Biol, 1996. 134(6): p. 1551-62.
79. Palecek, S. P., et al., Integrin-ligand binding properties govern cell migration speed through cell-substratum adhesiveness. Nature, 1997. 385(6616): p. 537-40.
80. de Bruyn, K. M., et al., The small GTPase Rap1 is required for Mn(2+)- and antibody-induced LEA-1- and VLA-4-mediated cell adhesion. J Biol Chem, 2002. 277(33): p. 29468-76.
81. Lefort, C. T., et al., Outside-in signal transmission by conformational changes in integrin Mac-1. J Immunol, 2009. 183(10): p. 6460-8.
82. Springer, T., et al., Mac-1: a macrophage differentiation antigen identified by monoclonal antibody. Eur J Immunol, 1979. 9(4): p. 301-6.
83. De Vriese, A. S., et al., The role of selectins in glomerular leukocyte recruitment in rat anti-glomerular basement membrane glomerulonephritis. J Am Soc Nephrol, 1999. 10(12): p. 2510-7.

1. Any one of the compounds listed in Table 1 or Table 2 or a derivative, salt, or ester thereof that augment beta2 integrin activity.

2. The compound of claim 1, wherein the compound is listed in Table 2.

3. The compound of claim 1, wherein the beta2 integrin is CD11b/CD18.

4. The compound of claim 1, wherein the beta2 integrin is CD11b^{E320A}/CD18.

5. The compound of claim 1, wherein the beta2 integrin is CD11c/CD18.

6. The compound of claim 1, wherein the compound binds to an αA-domain of a beta2 integrin.

7. The compound of claim 1, wherein the compound binds to the αA-domain of a CD11b integrin.

8. The compound of claim 1, wherein the compound binds to an αA-domain of a CD11c integrin.

9. A pharmaceutical composition comprising one or more of the compounds of claim 1 and one or more pharmaceutically acceptable excipients.

10-12. (canceled)

13. A method for identifying agonist compounds of beta2 integrin, the method comprising:
(a) contacting cells with a compound on a substrate treated with fibrinogen;
(b) physically repositioning the substrate such that non-adherent cells move away from the substrate by the action of gravity; and
(c) detecting adherent cells on the substrate.

14. The method of claim 13, further comprising the step of:
(d) quantifying the adherent cells on the substrate.

15. The method of claim 13, wherein the cells are K562 cells expressing integrin CD11b/CD18, CD11bE320A/CD18, or CD11c/CD18.

16. The method claim 13, wherein the solution further comprises Mg²⁺, Ca²⁺, or a mixture thereof.

17. The method of claim 13, further comprising removing the solution from the substrate after physically repositioning the substrate.

18. The method of claim 13, wherein removing the solution from the substrate comprises contacting adhered cells with a fixative and washing the substrate.

19. The method of claim 13, wherein the fixative comprises formaldehyde.

20. The method of claim 13, which is conducted without contacting the substrate with an additional liquid to wash or rinse the substrate.

21. The method of claim 13, wherein detecting adherent cells comprises measuring the viability of the adherent cells or imaging the adherent cells.

22. A beta2 integrin agonist compound identified by the method of claim 13.

23-28. (canceled)


SEQUENCE LISTING (4 sequences)

SEQ ID NO: 1 - protein, 1152 aa, Homo sapiens
MALRVLLLTALTLCHGFNLDTENAMTFQENARGFGQSVVQLQGSRVVVGAPQEIVAANQRGSLYQCDYSTGSCEPIRLQVPVEAVNMSLGLSLAATTSPPQLLACGPTVHQTCSENTYVKGLCFLFGSNLRQQPQKFPEALRGCPQEDSDIAFLIDGSGSIIPHDFRRMKEFVSTVMEQLKKSKTLFSLMQYSEEFRIHFTFKEFQNNPNPRSLVKPITQLLGRTHTATGIRKVVRELFNITNGARKNAFKILVVITDGEKFGDPLGYEDVIPEADREGVIRYVIGVGDAFRSEKSRQELNTIASKPPRDHVFQVNNFEALKTIQNQLREKIFAIEGTQTGSSSSFEHEMSQEGFSAAITSNGPLLSTVGSYDWAGGVFLYTSKEKSTFINMTRVDSDMNDAYLGYAAAIILRNRVQSLVLGAPRYQHIGLVAMFRQNTGMWESNANVKGTQIGAYFGASLCSVDVDSNGSTDLVLIGAPHYYEQTRGGQVSVCPLPRGRARWQCDAVLYGEQGQPWGRFGAALTVLGDVNGDKLTDVAIGAPGEEDNRGAVYLFHGTSGSGISPSHSQRIAGSKLSPRLQYFGQSLSGGQDLTMDGLVDLTVGAQGHVLLLRSQPVLRVKAIMEFNPREVARNVFECNDQVVKGKEAGEVRVCLHVQKSTRDRLREGQIQSVVTYDLALDSGRPHSRAVFNETKNSTRRQTQVLGLTQTCETLKLQLPNCIEDPVSPIVLRLNFSLVGTPLSAFGNLRPVLAEDAQRLFTALFPFEKNCGNDNICQDDLSITFSFMSLDCLVVGGPREFNVTVTVRNDGEDSYRTQVTFFFPLDLSYRKVSTLQNQRSQRSWRLACESASSTEVSGALKSTSCSINHPIFPENSEVTFNITFDVDSKASLGNKLLLKANVTSENNMPRTNKTEFQLELPVKYAVYMVVTSHGVSTKYLNFTASENTSRVMQHQYQVSNLGQRSLPISLVFLVPVRLNQTVIWDRPQVTFSENLSSTCHTKERLPSHSDFLAELRKAPVVNCSIAVCQRIQCDIPFFGIQEEFNATLKGNLSFDWYIKTSHNHLLIVSTAEILFNDSVFTLLPGQGAFVRSQTETKVEPFEVPNPLPLIVGSSVGGLLLLALITAALYKLGFFKRQYKDMMSEGGPPGAEPQ

SEQ ID NO: 2 - protein, 1152 aa, Artificial Sequence
  Site Directed Mutation E320A
  MISC_FEATURE: E320A
MALRVLLLTALTLCHGFNLDTENAMTFQENARGFGQSVVQLQGSRVVVGAPQEIVAANQRGSLYQCDYSTGSCEPIRLQVPVEAVNMSLGLSLAATTSPPQLLACGPTVHQTCSENTYVKGLCFLFGSNLRQQPQKFPEALRGCPQEDSDIAFLIDGSGSIIPHDFRRMKEFVSTVMEQLKKSKTLFSLMQYSEEFRIHFTFKEFQNNPNPRSLVKPITQLLGRTHTATGIRKVVRELFNITNGARKNAFKILVVITDGEKFGDPLGYEDVIPEADREGVIRYVIGVGDAFRSEKSRQELNTIASKPPRDHVFQVNNFEALKTIQNQLRAKIFAIEGTQTGSSSSFEHEMSQEGFSAAITSNGPLLSTVGSYDWAGGVFLYTSKEKSTFINMTRVDSDMNDAYLGYAAAIILRNRVQSLVLGAPRYQHIGLVAMFRQNTGMWESNANVKGTQIGAYFGASLCSVDVDSNGSTDLVLIGAPHYYEQTRGGQVSVCPLPRGRARWQCDAVLYGEQGQPWGRFGAALTVLGDVNGDKLTDVAIGAPGEEDNRGAVYLFHGTSGSGISPSHSQRIAGSKLSPRLQYFGQSLSGGQDLTMDGLVDLTVGAQGHVLLLRSQPVLRVKAIMEFNPREVARNVFECNDQVVKGKEAGEVRVCLHVQKSTRDRLREGQIQSVVTYDLALDSGRPHSRAVFNETKNSTRRQTQVLGLTQTCETLKLQLPNCIEDPVSPIVLRLNFSLVGTPLSAFGNLRPVLAEDAQRLFTALFPFEKNCGNDNICQDDLSITFSFMSLDCLVVGGPREFNVTVTVRNDGEDSYRTQVTFFFPLDLSYRKVSTLQNQRSQRSWRLACESASSTEVSGALKSTSCSINHPIFPENSEVTFNITFDVDSKASLGNKLLLKANVTSENNMPRTNKTEFQLELPVKYAVYMVVTSHGVSTKYLNFTASENTSRVMQHQYQVSNLGQRSLPISLVFLVPVRLNQTVIWDRPQVTFSENLSSTCHTKERLPSHSDFLAELRKAPVVNCSIAVCQRIQCDIPFFGIQEEFNATLKGNLSFDWYIKTSHNHLLIVSTAEILFNDSVFTLLPGQGAFVRSQTETKVEPFEVPNPLPLIVGSSVGGLLLLALITAALYKLGFFKRQYKDMMSEGGPPGAEPQ

SEQ ID NO: 3 - protein, 1169 aa, Homo sapiens
MTRTRAALLLFTALATSLGFNLDTEELTAFRVDSAGFGDSVVQYANSWVVVGAPQKITAANQTGGLYQCGYSTGACEPIGLQVPPEAVNMSLGLSLASTTSPSQLLACGPTVHHECGRNMYLTGLCFLLGPTQLTQRLPVSRQECPRQEQDIVFLIDGSGSISSRNFATMMNFVRAVISQFQRPSTQFSLMQFSNKFQTHFTFEEFRRSSNPLSLLASVHQLQGFTYTATAIQNVVHRLFHASYGARRDAAKILIVITDGKKEGDSLDYKDVIPMADAAGIIRYAIGVGLAFQNRNSWKELNDIASKPSQEHIFKVEDFDALKDIQNQLKEKIFAIEGTETTSSSSFELEMAQEGFSAVFTPDGPVLGAVGSFTWSGGAFLYPPNMSPTFINMSQENVDMRDSYLGYSTELALWKGVQSLVLGAPRYQHTGKAVIFTQVSRQWRMKAEVTGTQIGSYFGASLCSVDVDSDGSTDLVLIGAPHYYEQTRGGQVSVCPLPRGWRRWWCDAVLYGEQGHPWGRFGAALTVLGDVNGDKLTDVVIGAPGEEENRGAVYLFHGVLGPSISPSHSQRIAGSQLSSRLQYFGQALSGGQDLTQDGLVDLAVGARGQVLLLRTRPVLWVGVSMQFIPAEIPRSAFECREQVVSEQTLVQSNICLYIDKRSKNLLGSRDLQSSVTLDLALDPGRLSPRATFQETKNRSLSRVRVLGLKAHCENFNLLLPSCVEDSVTPITLRLNFTLVGKPLLAFRNLRPMLAADAQRYFTASLPFEKNCGADHICQDNLGISFSFPGLKSLLVGSNLELNAEVMVWNDGEDSYGTTITFSHPAGLSYRYVAEGQKQGQLRSLHLTCDSAPVGSQGTWSTSCRINHLIFRGGAQITFLATFDVSPKAVLGDRLLLTANVSSENNTPRTSKTTFQLELPVKYAVYTVVSSHEQFTKYLNFSESEEKESHVAMHRYQVNNLGQRDLPVSINFWVPVELNQEAVWMDVEVSHPQNPSLRCSSEKIAPPASDFLAHIQKNPVLDCSIAGCLRFRCDVPSFSVQEELDFTLKGNLSFGWVRQILQKKVSVVSVAEITFDTSVYSQLPGQEAFMRAQTTTVLEKYKVHNPTPLIVGSSIGGLLLLALITAVLYKVGFFKRQYKEMMEEANGQIAPENGTQTPSPPTPHYPQDNV

SEQ ID NO: 4 - protein, 1162 aa, Homo sapiens
MTFGTVLLLSVLASYHGFNLDVEEPTIFQEDAGGFGQSVVQFGGSRLVVGAPLEVVAANQTGRLYDCAAATGMCQPIPLHIRPEAVNMSLGLTLAASTNGSRLLACGPTLHRVCGENSYSKGSCLLLGSRWEIIQTVPDATPECPHQEMDIVFLIDGSGSIDQNDFNQMKGFVQAVMGQFEGTDTLFALMQYSNLLKIHFTFTQFRTSPSQQSLVDPIVQLKGLTFTATGILTVVTQLFHHKNGARKSAKKILIVITDGQKYKDPLEYSDVIPQAEKAGIIRYAIGVGHAFQGPTARQELNTISSAPPQDHVFKVDNFAALGSIQKQLQEKIYAVEGTQSRASSSFQHEMSQEGFSTALTMDGLFLGAVGSFSWSGGAFLYPPNMSPTFINMSQENVDMRDSYLGYSTELALWKGVQNLVLGAPRYQHTGKAVIFTQVSRQWRKKAEVTGTQIGSYFGASLCSVDVDSDGSTDLILIGAPHYYEQTRGGQVSVCPLPRGQRVQWQCDAVLRGEQGHPWGRFGAALTVLGDVNEDKLIDVAIGAPGEQENRGAVYLFHGASESGISPSHSQRIASSQLSPRLQYFGQALSGGQDLTQDGLMDLAVGARGQVLLLRSLPVLKVGVAMRFSPVEVAKAVYRCWEEKPSALEAGDATVCLTIQKSSLDQLGDIQSSVRFDLALDPGRLTSRAIFNETKNPTLTRRKTLGLGIHCETLKLLLPDCVEDVVSPIILHLNFSLVREPIPSPQNLRPVLAVGSQDLFTASLPFEKNCGQDGLCEGDLGVTLSFSGLQTLTVGSSLELNVIVTVWNAGEDSYGTVVSLYYPAGLSHRRVSGAQKQPHQSALRLACETVPTEDEGLRSSRCSVNHPIFHEGSNGTFIVTFDVSYKATLGDRMLMRASASSENNKASSSKATFQLELPVKYAVYTMISRQEESTKYFNFATSDEKKMKEAEHRYRVNNLSQRDLAISINFWVPVLLNGVAVWDVVMEAPSQSLPCVSERKPPQHSDFLTQISRSPMLDCSIADCLQFRCDVPSFSVQEELDFTLKGNLSFGWVRETLQKKVLVVSVAEITFDTSVYSQLPGQEAFMRAQMEMVLEEDEVYNAIPIIMGSSVGALLLLALITATLYKLGFFKRHYKEMLEDKPEDTATFSGDDFSCVAPNVPLS